(19) United States
(12) Patent Application Publication
(10) Pub. No.: US 2019/0267545 A1
(43) Pub. Date: Aug. 29, 2019

(54) PHOTOELECTRIC CONVERSION ELEMENT, AND IMAGING ELEMENT AND IMAGING APPARATUS INCLUDING THE SAME

(71) Applicant: CANON KABUSHIKI KAISHA, Tokyo (JP)

(72) Inventors: Tomona Yamaguchi, Tokyo (JP); Naoki Yamada, Inagi-shi (JP); Tetsuo Takahashi, Kawasaki-shi (JP); Jun Kamatani, Tokyo (JP); Yosuke Nishide, Kawasaki-shi (JP); Hirokazu Miyashita, Ebina-shi (JP); Satoru Shiobara, Hiratsuka-shi (JP); Hironobu Iwawaki, Yokohama-shi (JP); Hiroki Ohrui, Kawasaki-shi (JP); Masumi Itabashi, Yamato-shi (JP)

(21) Appl. No.: 16/405,874

(22) Filed: May 7, 2019

Related U.S. Application Data
(63) Continuation of application No. PCT/JP2017/039669, filed on Nov. 2, 2017.

(30) Foreign Application Priority Data
Nov. 11, 2016 (JP) 2016-220716

(51) Int. Cl. H01L 51/00; C07C 225/24; C07C 211/61; H01L 27/30; H01L 51/42; H01L 51/44

(52) U.S. Cl. CPC H01L 51/006; H01L 51/0046; H01L 51/0072; H01L 51/0058; C07C 225/24; C07C 2603/18; H01L 27/307; H01L 51/4273; H01L 51/448; H01L 51/0052; C07C 2602/08; C07C 211/61

(57) ABSTRACT

The present disclosure provides a photoelectric conversion element including a lower electrode, a photoelectric conversion layer, and an upper electrode in this order, wherein the photoelectric conversion layer includes a first organic compound and a second organic compound having a lower reduction potential than that of the first organic compound, the first organic compound has an emission lifetime of 1.1 ns or more in chloroform solution, and the first organic compound is an organic compound represented by Formula [1] according to claim 1, a fluoranthene derivative, or a metal complex.

### CROSS-REFERENCE TO RELATED APPLICATIONS

This application is a Continuation of International Patent Application No. PCT/JP2017/039669, filed Nov. 2, 2017, which claims the benefit of Japanese Patent Application No. 2016-220716, filed Nov. 11, 2016, both of which are hereby incorporated by reference herein in their entirety.

### TECHNICAL FIELD

The present invention relates to a photoelectric conversion element, and an imaging element and an imaging apparatus including the element.

### BACKGROUND ART

In recent years, photoelectric conversion elements using organic compounds in photoelectric conversion layers have been developed. A photoelectric conversion element is an element including a pair of electrodes and an organic photoelectric conversion layer arranged therebetween.

The photoelectric conversion element is an element converting optical information into electric information, and by utilizing this property, development of imaging elements has been promoted. Specifically, the imaging element is a solid-state imaging element having a structure in which a photoelectric conversion element is formed on a signal readout substrate.

In order to apply the photoelectric conversion element to practical use, there is room for improvement, such as a reduction in dark current and an improvement in photoelectric conversion efficiency. As an example thereof, various studies have been conducted for improving the photoelectric conversion efficiency of a photoelectric conversion element.

PTL 1 describes that a photoelectric conversion film including a p-type semiconductor layer and an n-type semiconductor layer has high photoelectric conversion efficiency by including fullerene or a fullerene derivative.

However, since the process of conversion from light energy to electric energy has not been investigated, energy that should be converted into electric energy is deactivated, and sufficient photoelectric conversion efficiency has not been obtained.

### CITATION LIST

### Patent Literature

PTL 1 Japanese Patent Laid-Open No. 2007-123707

### SUMMARY OF INVENTION

It is an object of the present invention to provide a photoelectric conversion element having high photoelectric conversion efficiency by using a photoelectric conversion layer including a specific organic compound having an exciton lifetime of at least a certain length.

Accordingly, the present invention provides a photoelectric conversion element including a lower electrode, a photoelectric conversion layer, and an upper electrode in this order. The photoelectric conversion layer includes a first organic compound and a second organic compound having a lower reduction potential than that of the first organic compound. The first organic compound has an emission lifetime of 1.1 ns or more in chloroform solution and is an organic compound represented by any one of the following Formulae [1] to [5].

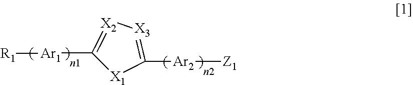

In Formula [1], R₁ represents a hydrogen atom, a halogen atom, a substituted or unsubstituted alkyl group, a substituted or unsubstituted alkoxy group, a substituted or unsubstituted aryl group, a substituted or unsubstituted heterocyclic group, a substituted or unsubstituted vinyl group, a substituted or unsubstituted amino group, or a cyano group.

n1 and n2 each represent an integer of 0 to 4. X₁ to X₃ each represent a nitrogen atom, a sulfur atom, an oxygen atom, or a carbon atom, and the carbon atom may have a substituent.

Ar₁ and Ar₂ are each independently selected from the group consisting of a substituted or unsubstituted aryl group and a substituted or unsubstituted heterocyclic group.

When there is more than one Ar₁, Ar₁'s may be the same or different, and when there is more than one Ar₂, Ar₂'s may be the same or different. When X₂ or X₃ is a carbon atom, Ar₁ and Ar₂ may form a ring by bonding to the carbon atom.

Z₁ represents a halogen atom, a cyano group, a substituted or unsubstituted heteroaryl group, or a substituent represented by any one of the following Formulae [1-1] to [1-9].

In Formulae [1-1] to [1-9], R₅₂₁ to R₅₈₈ are each independently selected from the group consisting of a hydrogen atom, a halogen atom, a substituted or unsubstituted alkyl group, a substituted or unsubstituted alkoxy group, a substituted or unsubstituted aryl group, a substituted or unsubstituted heterocyclic group, a substituted or unsubstituted vinyl group, a substituted or unsubstituted amino group, and a cyano group.

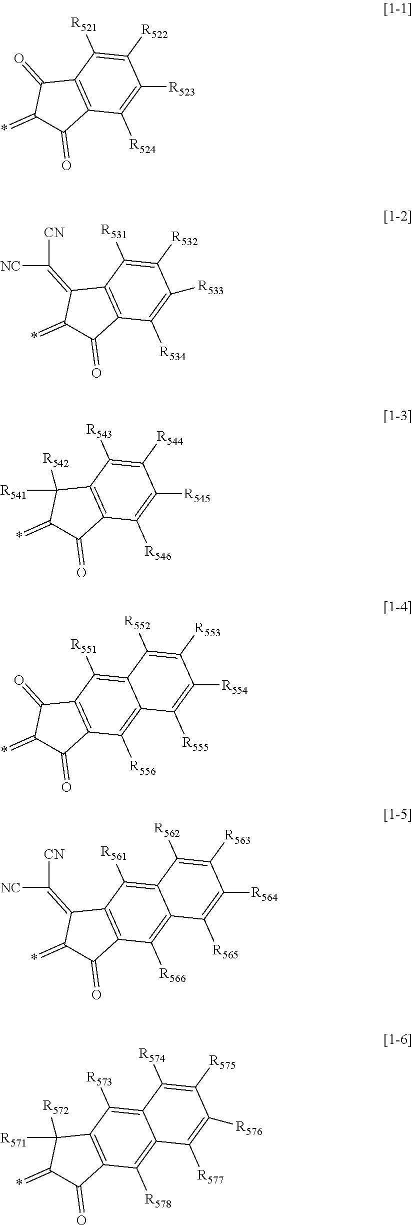

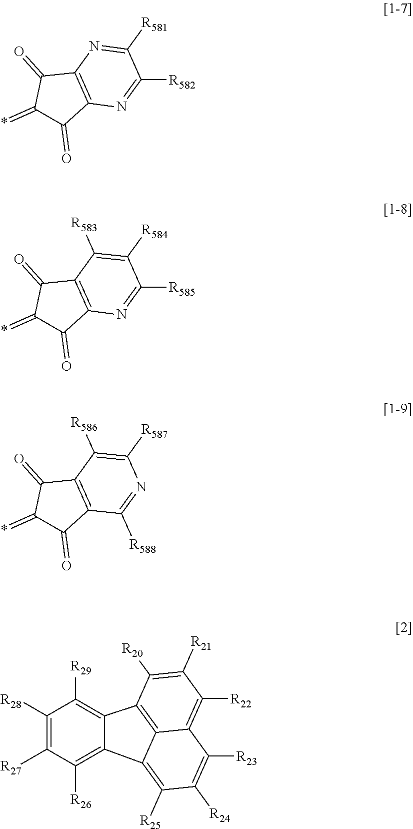

In Formula [2], R₂₀ to R₂₉ are each independently selected from the group consisting of a hydrogen atom, a halogen atom, a substituted or unsubstituted alkyl group, a substituted or unsubstituted alkoxy group, a substituted or unsubstituted aryl group, a substituted or unsubstituted heterocyclic group, a substituted or unsubstituted vinyl group, a substituted or unsubstituted amino group, and a cyano group. Adjacent two of R₂₀ to R₂₉ may form a ring by bonding to each other.

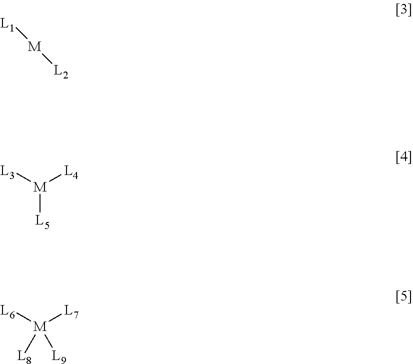

In Formulae [3] to [5], M represents a metal atom. The metal atom may have an oxygen atom or a halogen atom as a substituent.

L₁ to L₉ each represent a ligand coordinating to the metal M. The ligands are each a substituted or unsubstituted aryl group or a substituted or unsubstituted heterocyclic group, and adjacent two of L₁ to L₉ may form a ring by bonding to each other.

Further features of the present invention will become apparent from the following description of exemplary embodiments with reference to the attached drawings.

### BRIEF DESCRIPTION OF DRAWINGS

FIG. 1 is a diagram illustrating a photoinduced charge-separation process in a photoelectric conversion layer.

FIG. 2 is a diagram illustrating energy levels of a first organic compound and a second organic compound included in a photoelectric conversion layer.

FIG. 3 is a schematic cross-sectional view illustrating an example of a photoelectric conversion element according to the present invention.

FIG. 4 is a circuit diagram illustrating an example of a pixel including a photoelectric conversion device according to the present invention.

FIG. 5 is a diagram illustrating an imaging element including a photoelectric conversion element according to the present invention and a peripheral circuit thereof.

FIG. 6 is a decay curve of the emission intensity obtained in the emission lifetime measurement.

### DESCRIPTION OF EMBODIMENTS

The photoelectric conversion element according to the present invention has high photoelectric conversion efficiency due to the long lifetime of excitons generated by light absorption by a p-type organic semiconductor material in a photoelectric conversion layer. Furthermore, since the difference between the reduction potential of the p-type organic semiconductor material and the reduction potential of an n-type organic semiconductor material is large, the efficiency of electron transfer from the p-type organic semiconductor to the n-type organic semiconductor material is high, contributing to an improvement in the photoelectric conversion efficiency.

The photoelectric conversion element according to the present invention is an element including a lower electrode, a photoelectric conversion layer, and an upper electrode in this order. The element may be used by applying a voltage between these electrodes.

The photoelectric conversion layer includes a first organic compound and a second organic compound, and the first organic compound is an electron-donor material.

The first organic compound is the p-type organic semiconductor included in the photoelectric conversion layer. The first organic compound has a property of readily donating electrons. Specifically, among the two organic compounds, one having a lower oxidation potential is the first organic compound. That is, the first organic compound is an electron-donor material, and the second organic compound is an electron-acceptor material.

The photoelectric conversion layer preferably includes a bulk hetero layer. Consequently, the photoelectric conversion efficiency can be improved. By having the bulk hetero layer at an optimum mixing ratio, the photoelectric conversion layer can have increased electron mobility and hole mobility, and the photoelectric conversion element can have high photoresponse speed.

Regarding Emission Lifetime

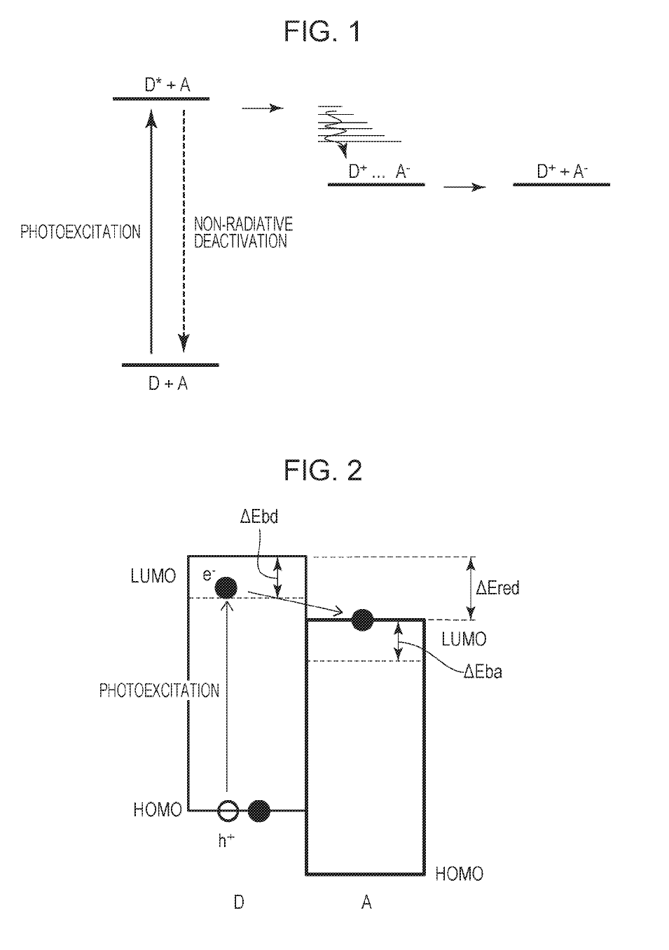

FIG. 1 is a diagram illustrating a photoinduced charge-separation process in a photoelectric conversion layer. The first organic compound (D) is raised to an excited state (D*) by irradiation with light. The generated D* interacts with the second organic compound (A) to be ionized into a charge transfer exciton, immediately followed by charge separation into D+ and A−, and each charge moves to the respective electrodes.

In order to make the above-mentioned process progress with high efficiency, it is preferred to increase the generation probability of the charge transfer excitons. When the time during which the first organic compound exists in the excited state (D*) is long, a larger number of molecules D* can approach the second organic compound (A) before the occurrence of radiative deactivation or non-radiative deactivation into the ground state. As a result, the generation probability of charge transfer excitons can be increased, and the photoelectric conversion element has high photoelectric conversion efficiency.

In the photoelectric conversion layer, in order to obtain a high probability of generating charge transfer excitons, it is preferable that the excited state of the first organic compound (D) continues for a long time. That is, a longer exciton lifetime of the first organic compound is preferred. In particular, an exciton lifetime of 1.1 ns or more gives high photoelectric conversion efficiency. An organic compound having a long exciton lifetime has a long emission lifetime.

In the present specification, the term “emission lifetime” refers to, when emission of light occurs in the process of molecular transition from the excited state to the ground state, the time until the ratio of the number of fluorescent molecules existing in the excited state to the initial number of the fluorescent molecules in the excited state becomes 1/e. That is, the molecule having a longer emission lifetime has a longer exciton lifetime. Accordingly, the first organic compound included in the photoelectric conversion element according to the present invention may have an exciton lifetime of 1.1 ns or more. Incidentally, e represents the Napier's constant.

The photoelectric conversion element according to the present invention exhibits excellent characteristics due to the structure represented by any one of Formulae [1] to [5], in addition to the long exciton lifetime of the first organic compound.

The first organic compound included in the photoelectric conversion layer is an organic compound represented by any one of the following Formulae [1] to [5]. It is particularly preferable that the first organic compound is an organic compound represented by Formula [1].

In Formula [1], R₁ represents a hydrogen atom, a halogen atom, a substituted or unsubstituted alkyl group, a substituted or unsubstituted alkoxy group, a substituted or unsubstituted aryl group, a substituted or unsubstituted heterocyclic group, a substituted or unsubstituted vinyl group, a substituted or unsubstituted amino group, or a cyano group.

n1 and n2 each represent an integer of 0 to 4.

X₁ to X₃ each represent a nitrogen atom, a sulfur atom, an oxygen atom, or a carbon atom, and the carbon atom may have a substituent.

Ar₁ and Ar₂ are each independently selected from the group consisting of a substituted or unsubstituted aryl group and a substituted or unsubstituted heterocyclic group. When there is more than one Ar₁, Ar₁'s may be the same or different, and when there is more than one Ar₂, Ar₂'s may be the same or different. When X₂ or X₃ is a carbon atom, Ar₁ and Ar₂ may form a ring by bonding to the carbon atom.

Z₁ represents a halogen atom, a cyano group, a substituted or unsubstituted heteroaryl group, or a substituent represented by any one of the following Formulae [1-1] to [1-9].

In Formulae [1-1] to [1-9], R₅₂₁ to R₅₈₈ are each independently selected from the group consisting of a hydrogen atom, a halogen atom, a substituted or unsubstituted alkyl group, a substituted or unsubstituted alkoxy group, a substituted or unsubstituted aryl group, a substituted or unsubstituted heterocyclic group, a substituted or unsubstituted vinyl group, a substituted or unsubstituted amino group, and a cyano group.

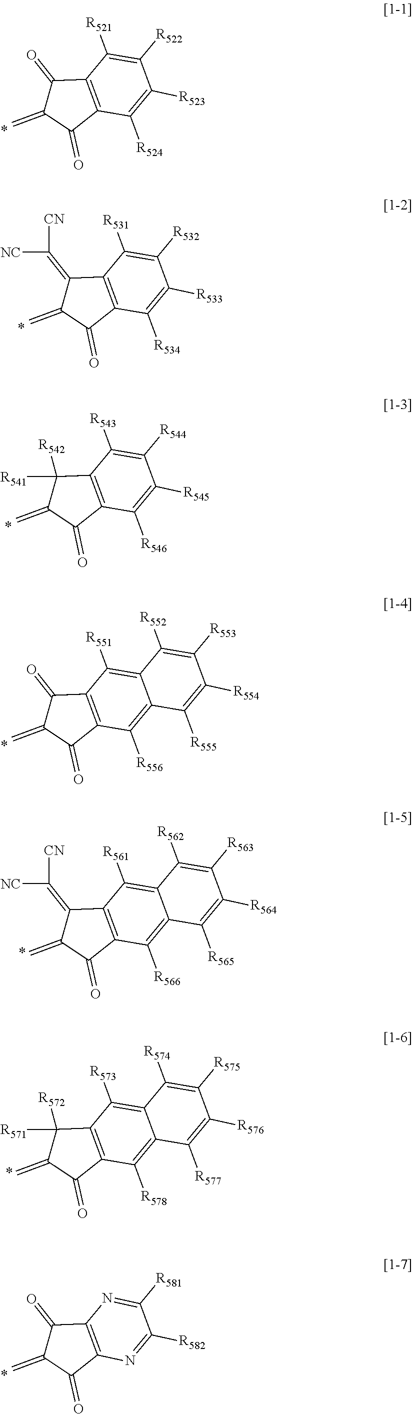

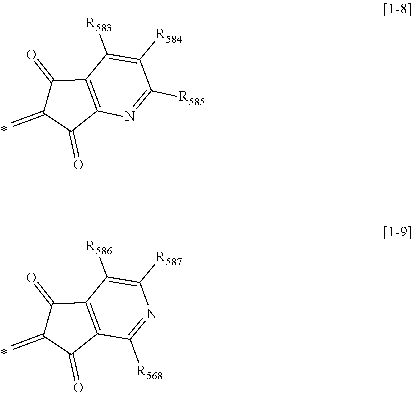

Among the organic compounds represented by Formula [1], Ar₁ is preferably a substituted or unsubstituted aryl group or a substituted or unsubstituted heterocyclic group. The heteroatom of the heterocyclic group is preferably nitrogen. X₁ is preferably a sulfur or oxygen atom. n1 is preferably 1, and n2 is preferably 0. Since n2 is 0, Are represents a single bond.

The first organic compound may be one represented the following Formula [2].

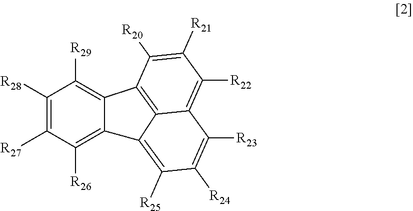

In Formula [2], R₂₀ to R₂₉ are each independently selected from the group consisting of a hydrogen atom, a halogen atom, a substituted or unsubstituted alkyl group, a substituted or unsubstituted alkoxy group, a substituted or unsubstituted aryl group, a substituted or unsubstituted heterocyclic group, a substituted or unsubstituted vinyl group, a substituted or unsubstituted amino group, and a cyano group. Adjacent two of R₂₀ to R₂₉ may form a ring by bonding to each other.

The first organic compound may be represented by any one of the following Formulae [3] to [5].

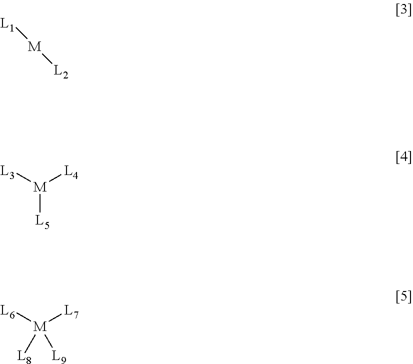

In Formulae [3] to [5], M represents a metal atom. The metal atom may have an oxygen atom or a halogen atom as a substituent.

L₁ to L₉ each represent a ligand coordinating to the metal M. The ligand is a substituted or unsubstituted aryl group or a substituted or unsubstituted heterocyclic group, and adjacent two of L₁ to L₉ may form a ring by bonding to each other.

Here, the ring structure to be formed is not restricted. For example, a five-membered ring may be formed by condensation, or a six-membered ring or a seven-membered ring may be formed by condensation. The condensed ring structure may be an aromatic ring structure or an aliphatic ring structure. Hereinafter, in the present specification, the term “may form a ring” is used as the same meaning unless otherwise specified.

In Formulae [3] to [5], when M is iridium, the compound is preferably a six-coordinated complex. When M is platinum, vanadium, cobalt, gallium, or titanium, the compound is preferably a four-coordinated complex. The complexes are stable at these coordination numbers.

More specifically, Formula [2] can be represented by any one of the following Formulae [11] to [27].

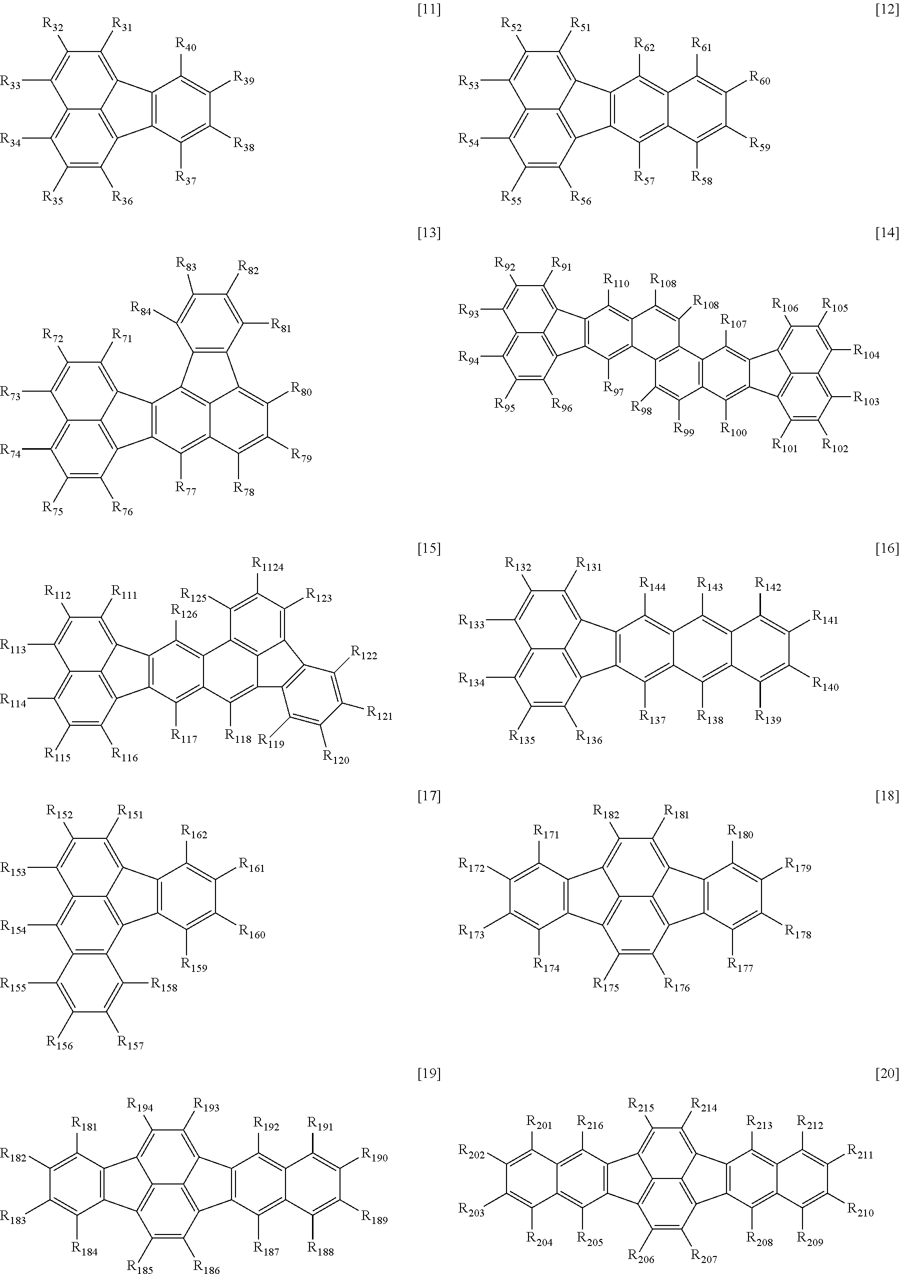

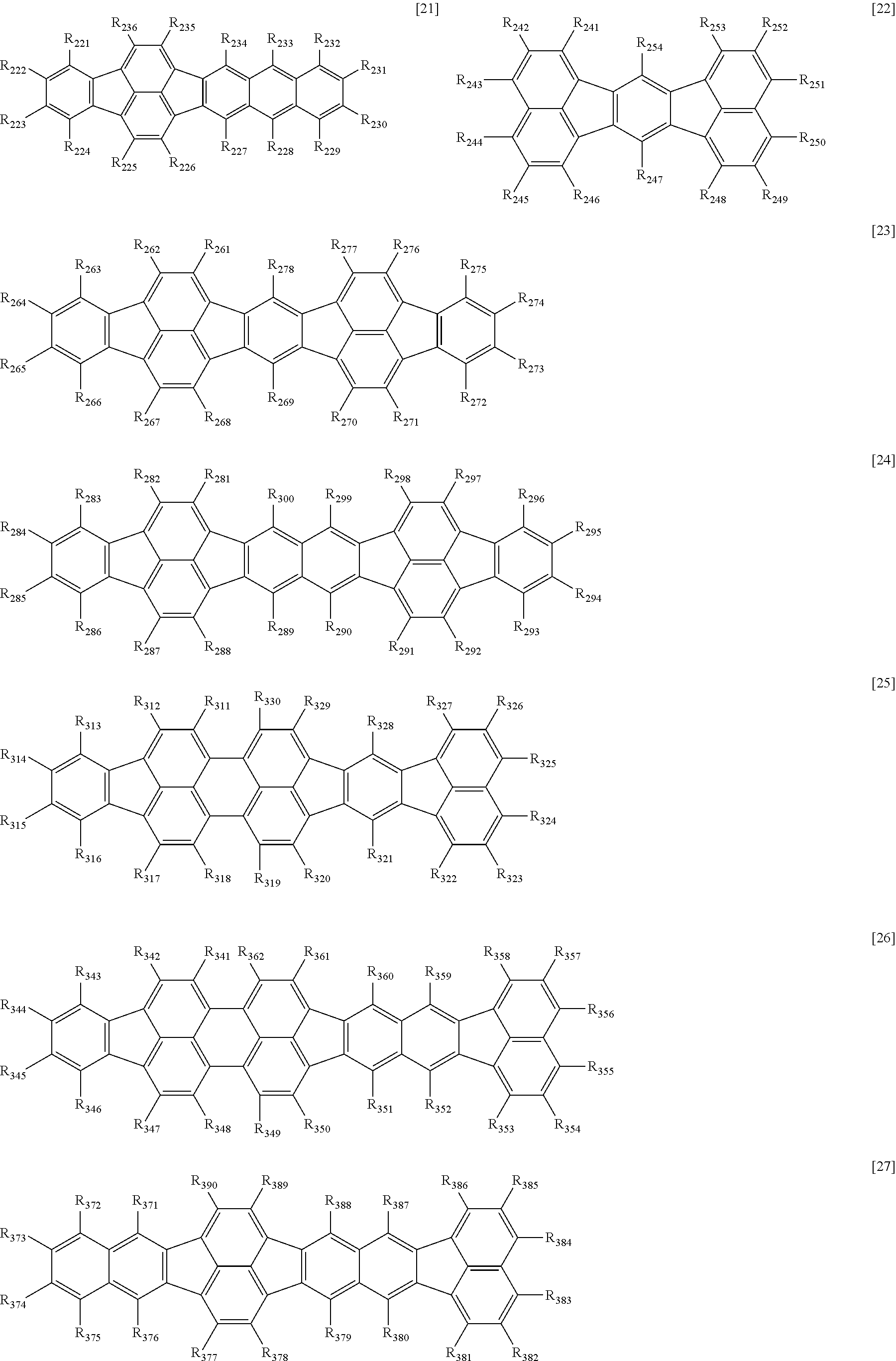

In Formulae [11] to [27], R₃₁ to R₃₉₀ are each independently selected from the group consisting of a hydrogen atom, a halogen atom, a substituted or unsubstituted alkyl group, a substituted or unsubstituted alkoxy group, a substituted or unsubstituted aryl group, a substituted or unsubstituted heterocyclic group, a substituted or unsubstituted vinyl group, a substituted or unsubstituted amino group, and a cyano group.

Examples of the substituent in Formulae [1] and [2], Formulae [1-1] to [1-9], and Formulae [11] to [27] are shown below.

Examples of the halogen atom include a fluorine atom, a chlorine atom, a bromine atom, and an iodine atom, and a fluorine atom is preferred.

The alkyl group is preferably an alkyl group having 1 to 10 carbon atoms. Examples thereof include a methyl group, an ethyl group, a n-propyl group, an isopropyl group, a n-butyl group, a tert-butyl group, a sec-butyl group, an octyl group, a 1-adamantyl group, and 2-adamantyl group. The alkyl group may be an alkyl group having 1 to 4 carbon atoms.

The alkoxy group is preferably an alkoxy group having 1 to 10 carbon atoms. Examples thereof include a methoxy group, an ethoxy group, a n-propoxy group, an isopropoxy group, a n-butoxy group, a tert-butoxy group, sec-butoxy group, and an octoxy group. The alkoxy group may be an alkoxy group having 1 to 4 carbon atoms.

The aryl group is preferably an aryl group having 6 to 20 carbon atoms. Examples thereof include a phenyl group, a naphthyl group, an indenyl group, a biphenyl group, a terphenyl group, a fluorenyl group, an anthracenyl group, a pyrenyl group, a fluoranthenyl group, and a perylenyl group. In particular, a phenyl group, a biphenyl group, a terphenyl group, a fluorenyl group, and a naphthyl group have low molecular weights and are preferred in consideration of sublimability of the compound.

The heterocyclic group is preferably a heterocyclic group having 3 to 15 carbon atoms. Examples thereof include a pyridyl group, a pyrazyl group, a triazyl group, a thienyl group, a furanyl group, a pyrrolyl group, an oxazolyl group, an oxadiazolyl group, a thiazolyl group, a thiadiazolyl group, a carbazolyl group, an acridinyl group, a phenanthrolyl group, a benzothiophenyl group, a dibenzothiophenyl group, a benzothiazolyl group, a benzazolyl group, and a benzopyrrolyl group. The heteroatom of the heterocyclic group is preferably nitrogen.

The amino group is preferably an amino group having an alkyl group or an aryl group as a substituent. Examples thereof include an N-methylamino group, an N-ethylamino group, an N,N-dimethylamino group, an N,N-diethylamino group, an N-methyl-N-ethylamino group, an N-benzoylamino group, an N-methyl-N-benzoylamino group, an N,N-dibenzoylamino group, an anilino group, an N,N-diphenylamino group, an N,N-dinaphthylamino group, an N,N-difluorenylamino group, an N-phenyl-N-tolylamino group, an N,N-ditolylamino group, an N-methyl-N-phenylamino group, an N,N-dianisolylamino group, an N-mesityl-N-phenylamino group, an N,N-dimesitylamino group, an N-phenyl-N-(4-tert-butylphenyl)amino group, and an N-phenyl-N-(4-trifluoromethylphenyl)amino group. Examples of the alkyl group or the aryl group of the amino group as the substituents are as shown in the examples of the substituent mentioned above.

Examples of the substituent of the alkyl group, the aryl group, the heterocyclic group, the amino group, the vinyl group, or the aryl group in Formulae [1] and [2], Formulae [1-1] to [1-9], and Formulae [11] to [27] include the following substituents. The substituents include an alkyl group having 1 to 4 carbon atoms, such as a methyl group, an ethyl group, a propyl group, and a butyl group; an aralkyl group, such as a benzyl group; an aryl group, such as a phenyl group and a biphenyl group; a heterocyclic group having a nitrogen atom as the heteroatom, such as a pyridyl group and a pyrrolyl group; an amino group, such as a dimethylamino group, a diethylamino group, a dibenzoylamino group, a diphenylamino group, and a ditolylamino group; an alkoxyl group, such as a methoxyl group, an ethoxyl group, a propoxyl group, and a phenoxyl group; a cyclic ketone group, such as a 1,3-indandionyl group, a 5-fluoro-1,3-indandionyl group, a 5,6-difluoro-1,3-indandionyl group, a 5,6-dicyano-1,3-indandionyl group, a 5-cyano-1,3-indandionyl group, a cyclopenta[b]naphthalene-1,3(2H)-dionyl group, a phenalene-1,3(2H)-dionyl group, and a 1,3-diphenyl-2,4,6(1H,3H,5H)-pyrimidinetrionyl group; a cyano group; and a halogen atom. The halogen atoms is, for example, fluorine, chlorine, bromine, or iodine, and a fluorine atom is preferred.

Examples of the ligands L₁ to L₉ in Formulae [3] to [5] are shown below.

The ligands L₁ to L₉ are each a ligand to which a plurality of substituents selected from substituted or unsubstituted aryl groups and substituted or unsubstituted heterocyclic groups are bonded.

Examples of the aryl group constituting the ligand include, but not limited to, a phenyl group, a naphthyl group, an indenyl group, a biphenyl group, a terphenyl group, a fluorenyl group, an anthracenyl group, a pyrenyl group, a fluoranthenyl group, and a perylenyl group.

Examples of the heterocyclic group constituting the ligand include, but not limited to, a pyridyl group, a pyrazyl group, a triazyl group, a thienyl group, a furanyl group, a pyrrolyl group, an oxazolyl group, an oxadiazolyl group, a thiazolyl group, a thiadiazolyl group, a carbazolyl group, an acridinyl group, a phenanthrolyl group, a benzothiophenyl group, a dibenzothiophenyl group, a benzothiazolyl group, a benzazolyl group, and a benzopyrrolyl group.

Examples of the substituent of the ligand in Formulae [3] and [5], i.e., examples of the substituent of the aryl group or the heterocyclic group include an alkyl group having 1 to 4 carbon atoms, such as a methyl group, an ethyl group, a propyl group, and a butyl group; an aralkyl group, such as a benzyl group; an aryl group, such as a phenyl group and a biphenyl group; a heterocyclic group having a nitrogen atom as the heteroatom, such as a pyridyl group and a pyrrolyl group; an amino group, such as a dimethylamino group, a diethylamino group, a dibenzoylamino group, a diphenylamino group, and a ditolylamino group; an alkoxyl group, such as a methoxyl group, an ethoxyl group, a propoxyl group, and a phenoxyl group; a cyclic ketone group, such as a 1,3-indandionyl group, a 5-fluoro-1,3-indandionyl group, a 5,6-difluoro-1,3-indandionyl group, a 5,6-dicyano-1,3-indandionyl group, a 5-cyano-1,3-indandionyl group, a cyclopenta[b]naphthalene-1,3(2H)-dionyl group, a phenalene-1,3(2H)-dionyl group, and a 1,3-diphenyl-2,4,6(1H,3H,5H)-pyrimidinetrionyl group; a cyano group; and a halogen atom. The halogen atom is, for example, fluorine, chlorine, bromine, or iodine, and a fluorine atom is preferred.

The ligand may include, for example, a hydroxy group or a carboxyl group as a substituent and may be bonded to the metal atom via the hydroxy group or the carboxyl group.

Formula [1] preferably has a structure represented by the following Formula [28].

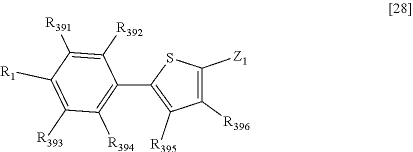

R₃₉₁ to R₃₉₆ are each independently selected from the group consisting of a hydrogen atom, a halogen atom, a substituted or unsubstituted alkyl group, a substituted or unsubstituted alkoxy group, a substituted or unsubstituted aryl group, a substituted or unsubstituted heterocyclic group, a substituted or unsubstituted vinyl group, a substituted or unsubstituted amino group, and a cyano group. Adjacent two of R₃₉₁ to R₃₉₆ may form a ring by bonding to each other. In particular, R₃₉₄ and R₃₉₅ preferably form a ring by bonding to each other.

The organic compound represented by Formula [28] is a material having strong absorption at the absorption peak wavelength of 522 nm or more and 600 nm or less. Having an absorption peak in this wavelength region is that the photoelectric conversion layer has a panchromatic property, as described above, and is therefore preferred.

The first organic compound preferably has an absorption wavelength in the visible region of 450 nm or more and 700 nm or less. In order that the photoelectric conversion layer obtains a panchromatic absorption band, it is particularly preferable that the absorption peak wavelength is 500 nm or more and 650 nm or less. The compound having an absorption peak wavelength in this region also has absorption in a region close to the region, i.e., the blue region of 450 nm or more and 470 nm or less and the red region of 600 nm or more and 630 nm or less, and the panchromatic property is therefore improved.

The weight rate of the first organic compound in the photoelectric conversion layer is preferably less than 35 wt % and more preferably 27.5 wt % or less when the total weight of the first organic compound and the second organic compound is defined as 100 wt %.

Regarding ΔEred

FIG. 2 is a diagram illustrating energy levels of the first organic compound (A) and the second organic compound (D). HOMO and LUMO in FIG. 2 are the highest occupied molecular orbital and the lowest unoccupied molecular orbital, respectively. The broken line between HOMO and LUMO in FIG. 2 indicates the excited level.

ΔEred is the difference between the reduction potential of the second organic compound and the reduction potential of the first organic compound, and is an energy gap defined by the following Expression (A). The ΔEred preferably satisfies the following Expression (B).

ΔEred=(reduction potential of second organic compound)−(reduction potential of first organic compound) (A)

ΔEred≥0.32 [V] (B)

0.32≤ΔEred≤0.65 [V] (C)

The reduction potential (Ered) is a potential at which a compound is reduced. That is, the reduction potential is chemically an anion radical state with one extra electron and is a potential energy for obtaining a free electron.

The reduction potential of the first organic compound (D) corresponds to the LUMO of the first organic compound (D), and the reduction potential of the second organic compound (A) corresponds to the LUMO of the second organic compound (A).

When the ΔEred is large, electron transfer from the second organic compound to the first organic compound can be prevented. Occurrence of electron transfer from the second organic compound to the first organic compound causes a risk of not achieving desired charge separation.

Irradiation of the first organic compound (D) with light excites an electron from the ground state to an excited state. The electron is transferred to the LUMO of the second organic compound (A), resulting in charge separation. When the LUMO of the first organic compound (D) is low, the energy of the excited level of the first organic compound (D) is near the energy of the LUMO of the second organic compound (A).

Since electron transfer tends to occur at energy levels with small energy difference, there is a possibility of electron transfer from the first organic compound to the second organic compound. As a result, charge separation does not occur, resulting in difficulty of obtaining a desired function. Accordingly, a higher LUMO of the first organic compound is preferred.

In addition, when the excited level of the first organic compound (D) is lower than the LUMO of the second organic compound (A), electron transfer is less likely to occur. When the LUMO of the first organic compound is high, the excited level of the first organic compound also tends to be high. It is preferable that the excited level of the first organic compound is higher than the LUMO of the second organic compound because of a high LUMO of the first organic compound.

Accordingly, a high ΔEred is preferred for causing charge separation with high efficiency. It is further preferred that the ΔEred is in a specific range. Specifically, the ΔEred preferably satisfies Expression (B) and more preferably satisfies Expression (C). Consequently, a photoelectric conversion element having further high photoelectric conversion efficiency can be obtained.

ΔEbd and ΔEba are the exciton binding energy of the first organic compound and the exciton binding energy of the second organic compound, respectively. The exciton binding energy is the difference between the LUMO and an excited level.

Photoelectric Conversion Element According to the Present Invention

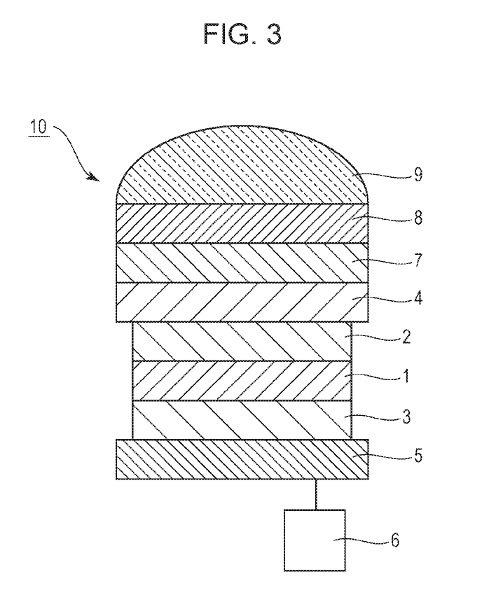

FIG. 3 is a schematic cross-sectional view illustrating an example of a photoelectric conversion element according to the embodiment. In the photoelectric conversion element, a photoelectric conversion layer 1 for converting light into a charge is arranged between a pair of electrodes, i.e., an anode electrode 4 and a cathode electrode 5. On the anode electrode, a protective layer 7, a wavelength selector 8, and a microlens 9 are arranged. The cathode electrode is connected to a readout circuit 6.

In the pair of electrodes, the electrode close to the substrate may be called a lower electrode, and the electrode far from the substrate may be called an upper electrode. The lower electrode may be the anode electrode or the cathode electrode. The lower electrode may be an electrode having high reflectance. The electrode may be constituted of a material with high reflectance or may include a reflecting layer in addition to an electrode layer.

The photoelectric conversion element according to the present invention may include a substrate. The substrate that can be used is, for example, a silicon substrate, a glass substrate, or a flexible substrate.

The cathode electrode of the photoelectric conversion element according to the present invention is an electrode that collects holes of a charge generated in the photoelectric conversion layer. In contrast, the anode electrode is an electrode that collects electrons in a charge generated in the photoelectric conversion layer. The materials constituting the cathode electrode and the anode electrode may be any materials that have transparency and high conductivity. The materials constituting the cathode electrode and the anode electrode may be the same or different.

Examples of the material for the electrodes include metals, metal oxides, metal nitrides, metal borides, organic conductive compounds, and mixtures thereof and, more specifically, conductive metal oxides, such as tin oxides doped with antimony or fluorine (ATO or FTO), tin oxide, zinc oxide, indium oxide, indium tin oxide (ITO), and indium zinc oxide (IZO); metals, such as gold, silver, chromium, nickel, titanium, tungsten, and aluminum, and conductive compounds, such as oxides and nitrides, of these metals (titanium oxide (TiN) is an example thereof); mixtures or laminates of these metals and conductive metal oxides; conductive inorganic materials, such as copper iodide and copper sulfide; conductive organic materials, such as polyaniline, polythiophene, and polypyrrole; and laminates of these materials and ITO or titanium nitride. Particularly preferred examples of the material for the electrode include titanium nitride, molybdenum nitride, tantalum nitride, and tungsten nitride.

The electrodes collecting holes or electrons of the photoelectric conversion element according to the present invention are electrodes collecting electrons or holes of a charge generated in the photoelectric conversion layer. In the configuration of an imaging element, the lower collection electrode may be a pixel electrode. Whether the pixel electrode is a cathode or an anode is determined depending on the element configuration or the underlying circuit configuration. For example, on a substrate, the order may be substrate/anode electrode/photoelectric conversion layer/cathode electrode may be arranged in this order, or substrate/cathode electrode/photoelectric conversion layer/anode electrode may be arranged in this order.

The method for forming an electrode can be appropriately selected in consideration of suitability with the electrode material. Specifically, the electrode can be formed by a wet system, such as a printing system and a coating system; a physical system, such as a vacuum vapor deposition method, a sputtering method, and an ion plating method; or a chemical system, such as a CVD and plasma CVD method.

An electrode of ITO can be formed by a method, such as an electron beam method, a sputtering method, a resistance heating deposition method, a chemical reaction method (e.g., sol-gel method), or application of an indium tin oxide dispersion. Furthermore, the formed ITO can be subjected to, for example, UV/ozone treatment or plasma treatment. In an electrode of TiN, a variety of methods represented by a reactive sputtering method is used, and the TiN can be subjected to, for example, annealing treatment, UV-ozone treatment, or plasma treatment.

The photoelectric conversion layer may include an organic compound in addition to those represented by Formulae [1] to [5]. For example, triarylamine compounds, pyran compounds, quinacridone compounds, benzidine compounds, pyrazoline compounds, styrylamine compounds, hydrazone compounds, triphenylmethane compounds, carbazole compounds, polysilane compounds, thiophene compounds, phthalocyanine compounds, cyanine compounds, merocyanine compounds, oxonol compounds, polyamine compounds, indole compounds, pyrrole compounds, pyrazole compounds, polyarylene compounds, condensed aromatic carbon ring compounds (naphthalene derivatives, anthracene derivatives, phenanthrene derivatives, tetracene derivatives, pyrene derivatives, perylene derivatives, and fluoranthene derivatives), and metal complexes including nitrogen-containing heterocyclic compounds as ligands can be used. In particular, triarylamine compounds, pyran compounds, quinacridone compound, pyrrole compounds, phthalocyanine compounds, merocyanine compounds, and condensed aromatic carbon ring compounds are preferred.

The fluoranthene derivative is a compound having a fluoranthene skeleton in the chemical structural formula. Compounds in which a condensed ring is added to the fluoranthene skeleton are also included. That is, the fluoranthene derivative means a compound of which the chemical structural formula includes a fluoranthene skeleton. This also applies to other derivatives, i.e., naphthalene derivatives, anthracene derivatives, phenanthrene derivatives, tetracene derivatives, pyrene derivatives, and perylene derivatives.

The photoelectric conversion layer may include fullerene or a fullerene derivative as the second organic compound. The fullerene or fullerene derivative may function as an n-type organic semiconductor.

The fullerene or fullerene derivative molecules are connected to one another in the photoelectric conversion layer to form an electron transporting path. Accordingly, the electron transport property is improved, and the rapid response property of the photoelectric conversion element is improved.

The weight rate of the fullerene or fullerene derivative is preferably 40 wt % or more and 85 wt % or less when the total of the first organic compound and the second organic compound is defined as 100 wt %.

Examples of the fullerene or fullerene derivative include fullerene C60, fullerene C70, fullerene C76, fullerene C78, fullerene C80, fullerene C82, fullerene C84, fullerene C90, fullerene C96, fullerene C240, fullerene 540, mixed fullerene, and fullerene nanotubes.

The fullerene derivative may have a substituent. Examples of the substituent include an alkyl group, an aryl group, and a heterocyclic group.

The fullerene derivative is preferably fullerene C60.

The photoelectric conversion layer is preferably non-emissive. Non-emission means that the light emission quantum efficiency in the visible light region (wavelength: 400 to 730 nm) is 1% or less, preferably 0.5% or less, and more preferably 0.1% or less. If the light emission quantum efficiency of the photoelectric conversion layer is 1% or less, when the layer is applied to a sensor or an imaging element, the influence on the sensing performance or the imaging performance is small. Accordingly, the layer is preferable as an imaging element.

The photoelectric conversion element according to the present invention may further include a hole blocking layer between the anode electrode and the photoelectric conversion layer. The hole blocking layer is a layer for preventing holes from flowing from the anode electrode into the photoelectric conversion layer and preferably has a high ionization potential.

The photoelectric conversion element according to the present invention may further an electron blocking layer between the cathode electrode and the photoelectric conversion layer. The electron blocking layer is a layer for preventing electrons from flowing from the cathode electrode into the photoelectric conversion layer and preferably has low electron affinity or a low LUMO (lowest unoccupied molecular orbital).

The photoelectric conversion element may further include a sealing layer on the upper electrode. The material constituting the sealing layer is not particularly limited, but is an inorganic material, specifically, for example, silicon oxide, silicon nitride, silicon oxynitride, or an oxide of aluminum. Silicon oxide, silicon nitride, and silicon oxynitride can be formed by a sputtering method or a CVD method, and an oxide of aluminum can be formed by an atomic layer deposition (ALD) method.

The sealing performance of the sealing layer may be a moisture permeability of 10⁻⁵ g/m² day or less. The thickness of the sealing layer is not particularly limited, but is preferably 0.5 μm or more from the viewpoint of the sealing performance. On the other hand, a smaller thickness is preferred as long as the sealing performance is maintained, and a thickness of 1 μm or less is particularly preferred.

A thinner sealing layer is preferred because when it is used as an imaging element, the effect of reducing color mixture becomes higher with shortening the distance from the photoelectric conversion layer to a color filter.

When a photoelectric conversion element is produced, it is preferable to perform an annealing step. Although the annealing temperature is not limited, the annealing temperature conditions may be 150° C. or more and 190° C. or less. The annealing temperature is appropriately determined by a balance with the annealing time.

Examples of the first organic compound are shown below.

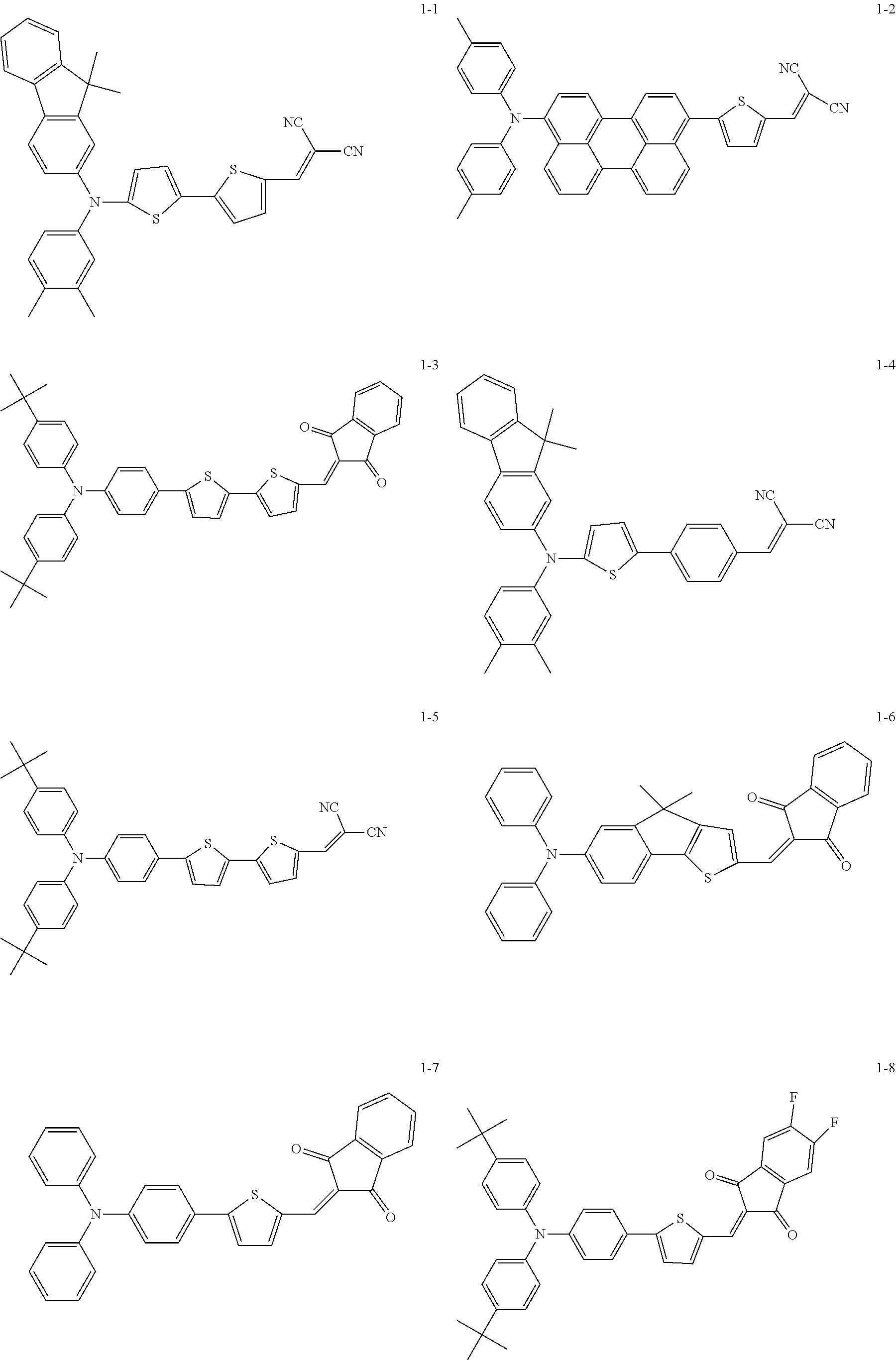

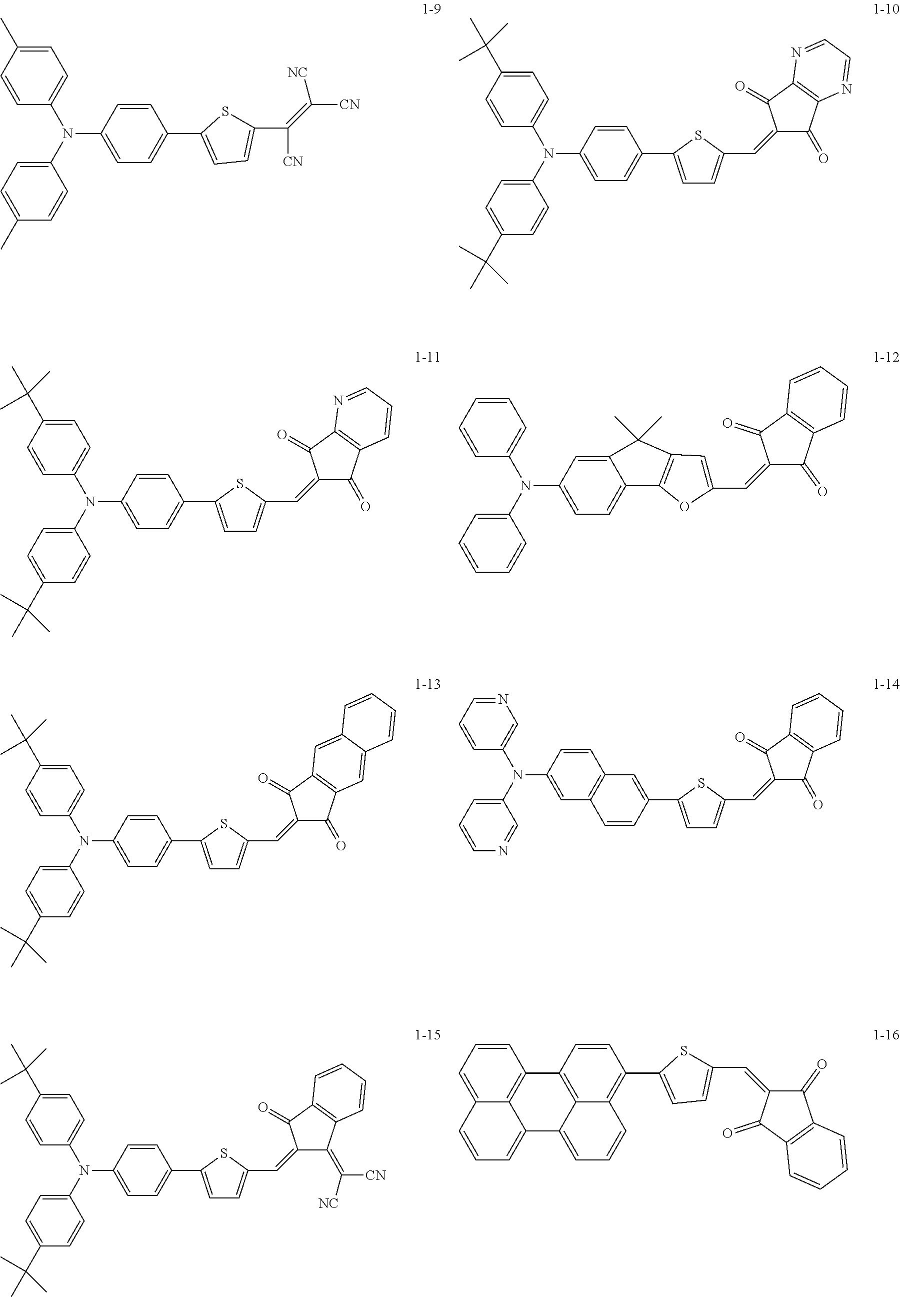

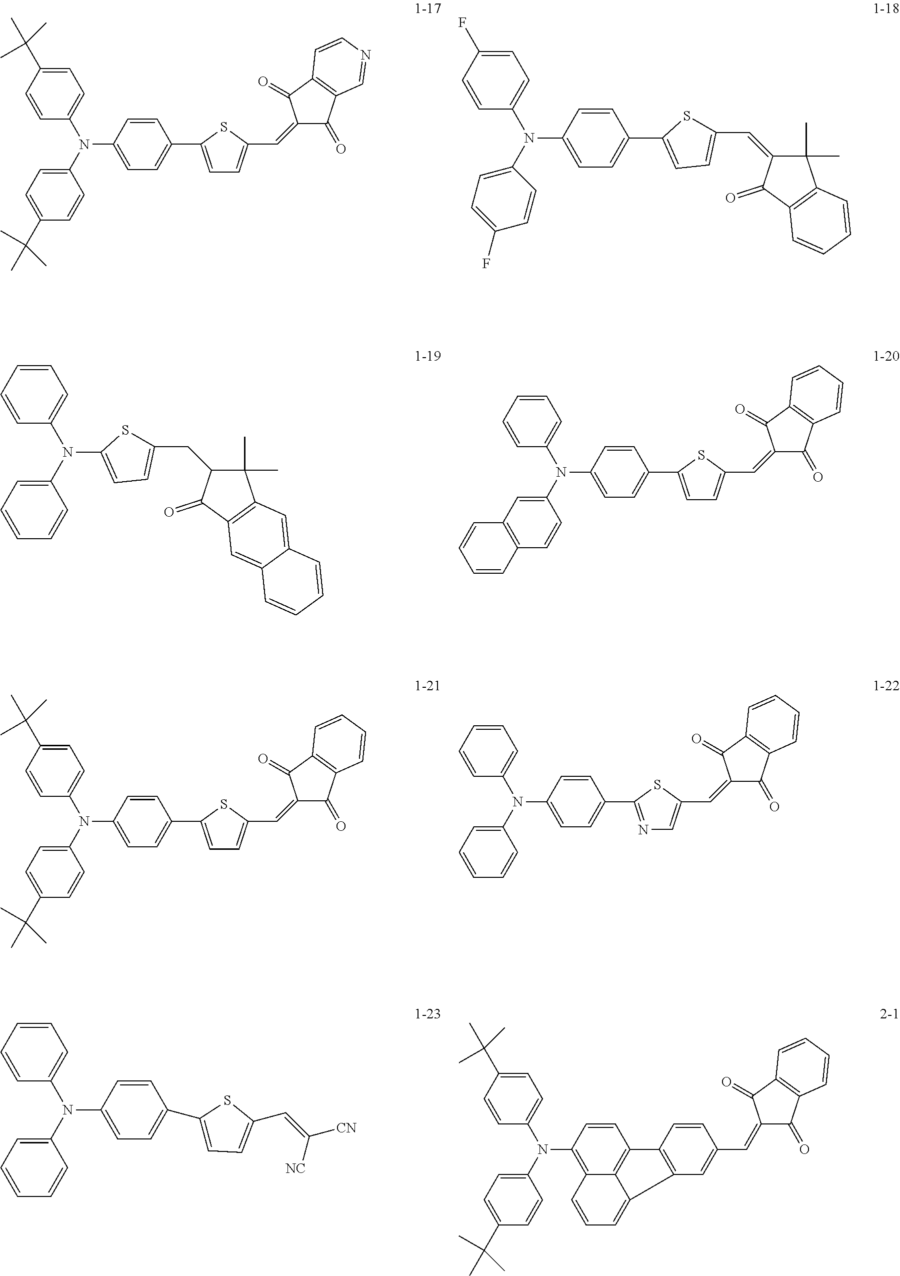

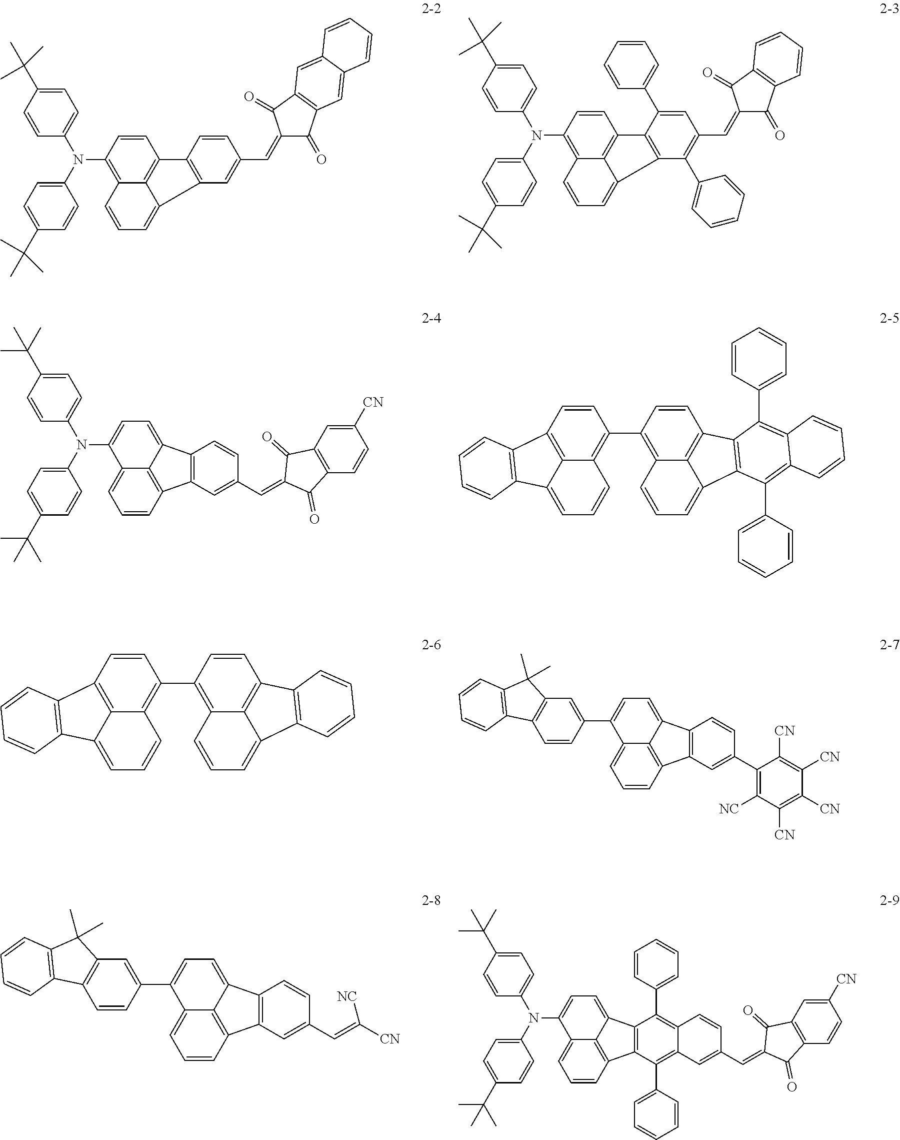

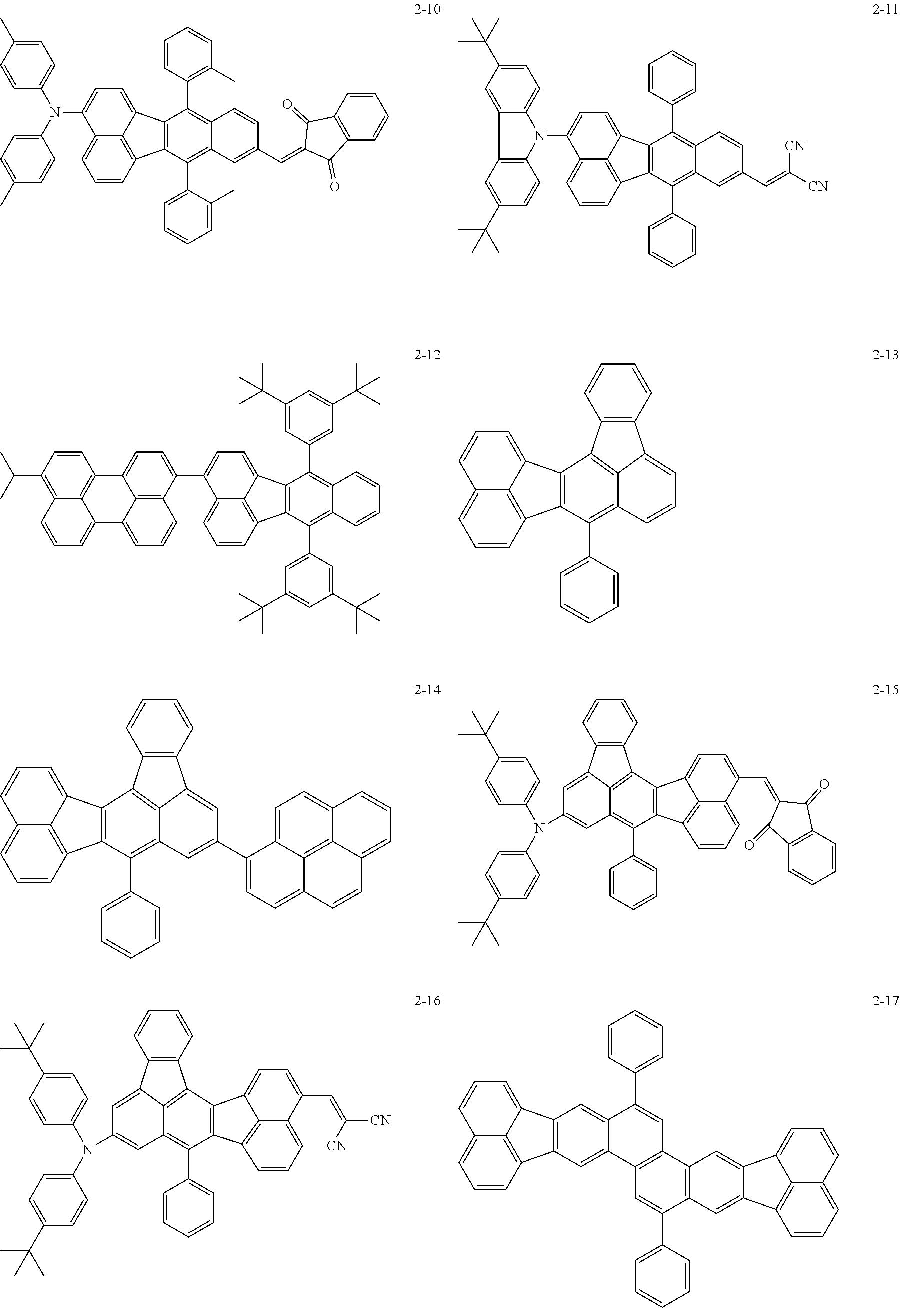

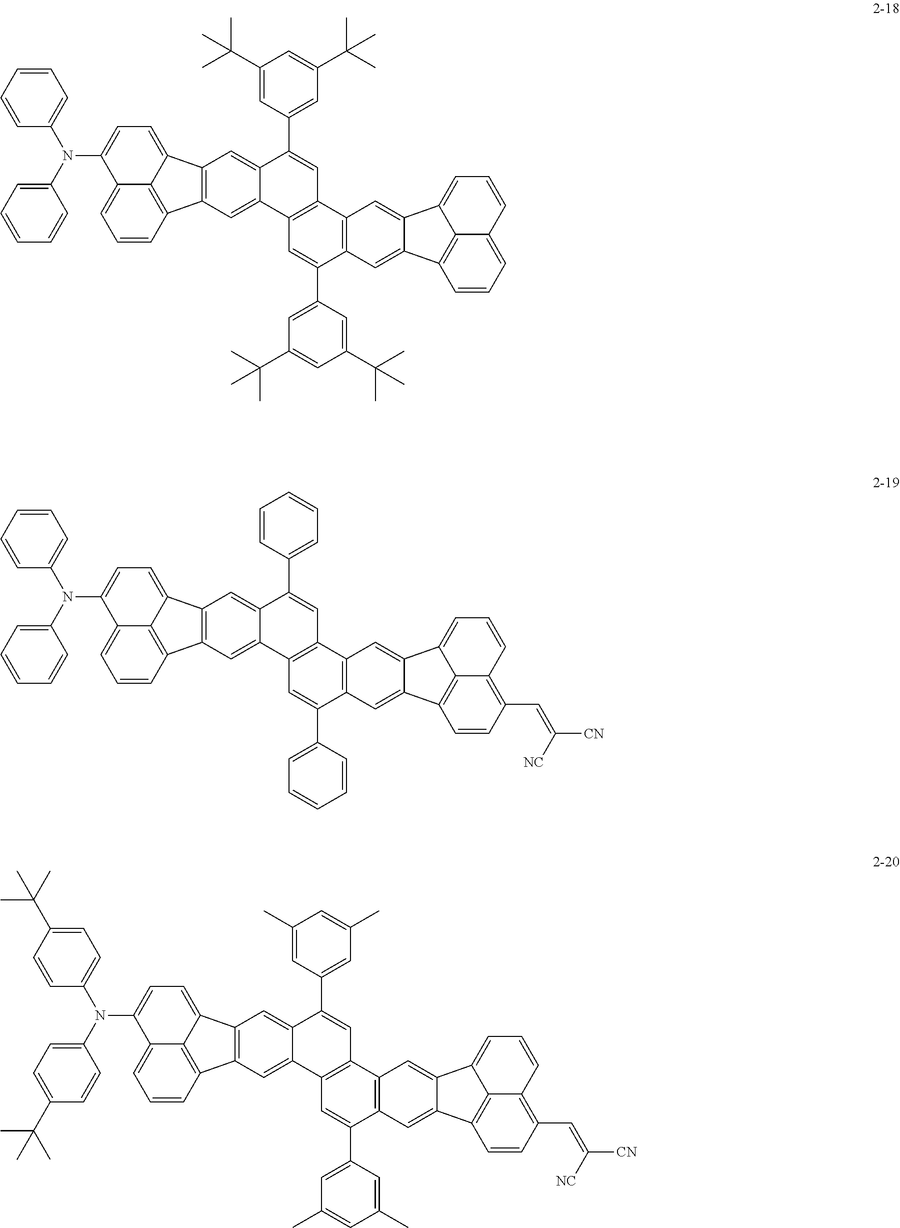 [SMILES: CC(C)(C)c1cc(-c2cc3c4cc5c(cc4c(-c4cc(C(C)(C)C)cc(C(C)(C)C)c4)cc3c3cc4c(cc23)-c2cccc3cccc-4c23)-c2ccc(N(c3ccccc3)c3ccccc3)c3cccc-5c23)cc(C(C)(C)C)c1 ; N#CC(C#N)=Cc1ccc2c3c(cccc13)-c1cc3c(cc1-2)c(-c1ccccc1)cc1c2cc4c(cc2c(-c2ccccc2)cc31)-c1ccc(N(c2ccccc2)c2ccccc2)c2cccc-4c12 ; Cc1cc(C)cc(-c2cc3c4cc5c(cc4c(-c4cc(C)cc(C)c4)cc3c3cc4c(cc23)-c2ccc(C=C(C#N)C#N)c3cccc-4c23)-c2ccc(N(c3ccc(C(C)(C)C)cc3)c3ccc(C(C)(C)C)cc3)c3cccc-5c23)c1]

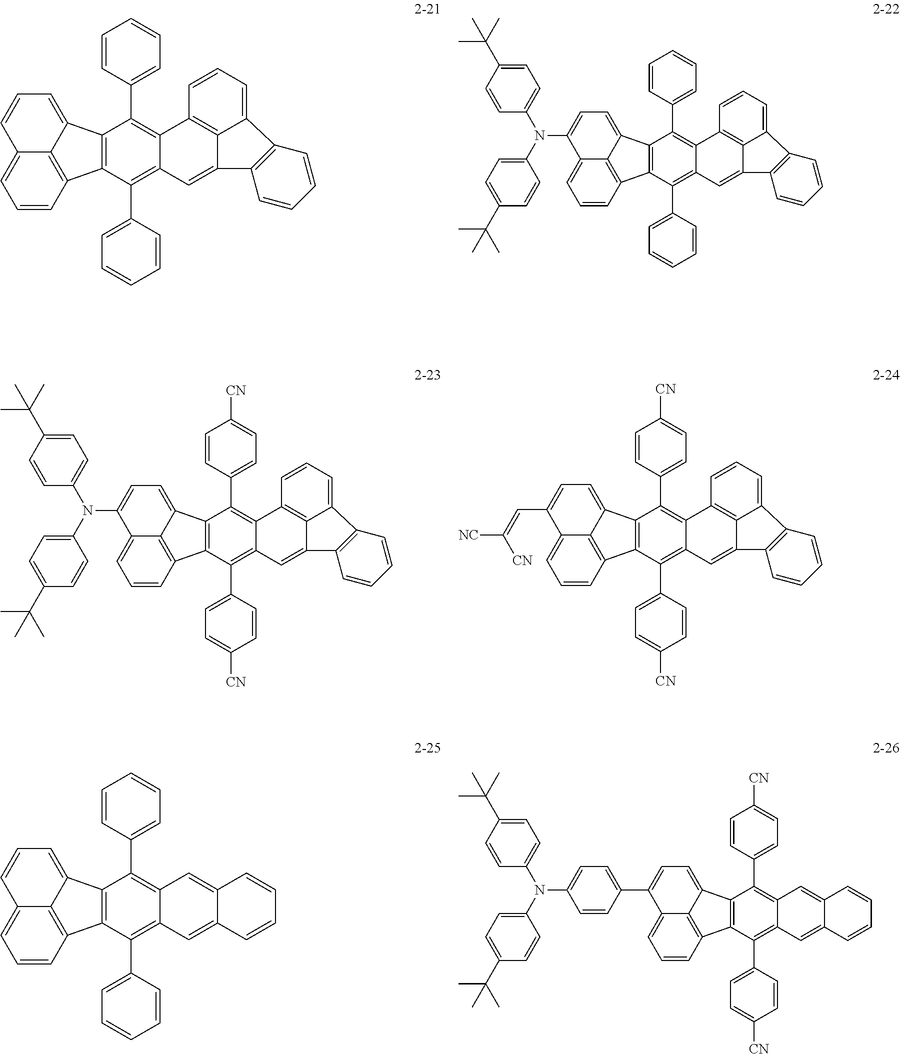

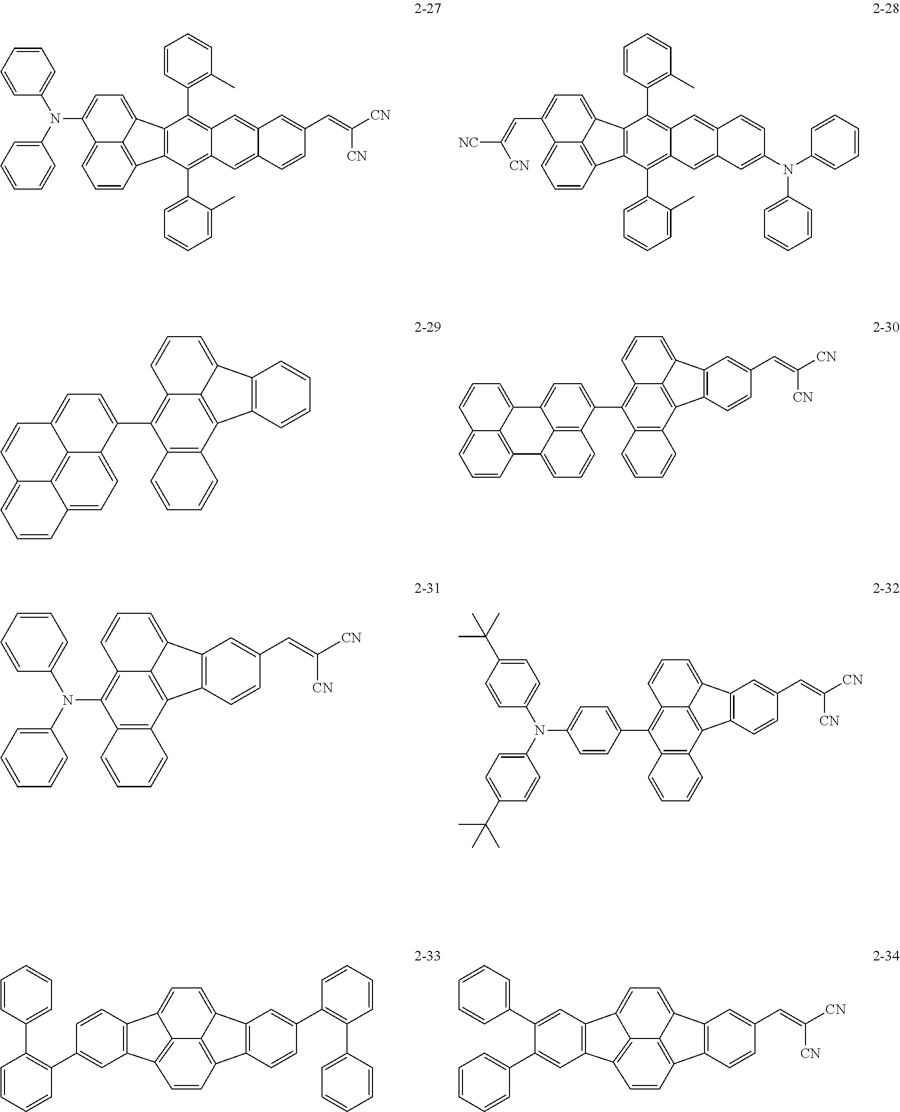

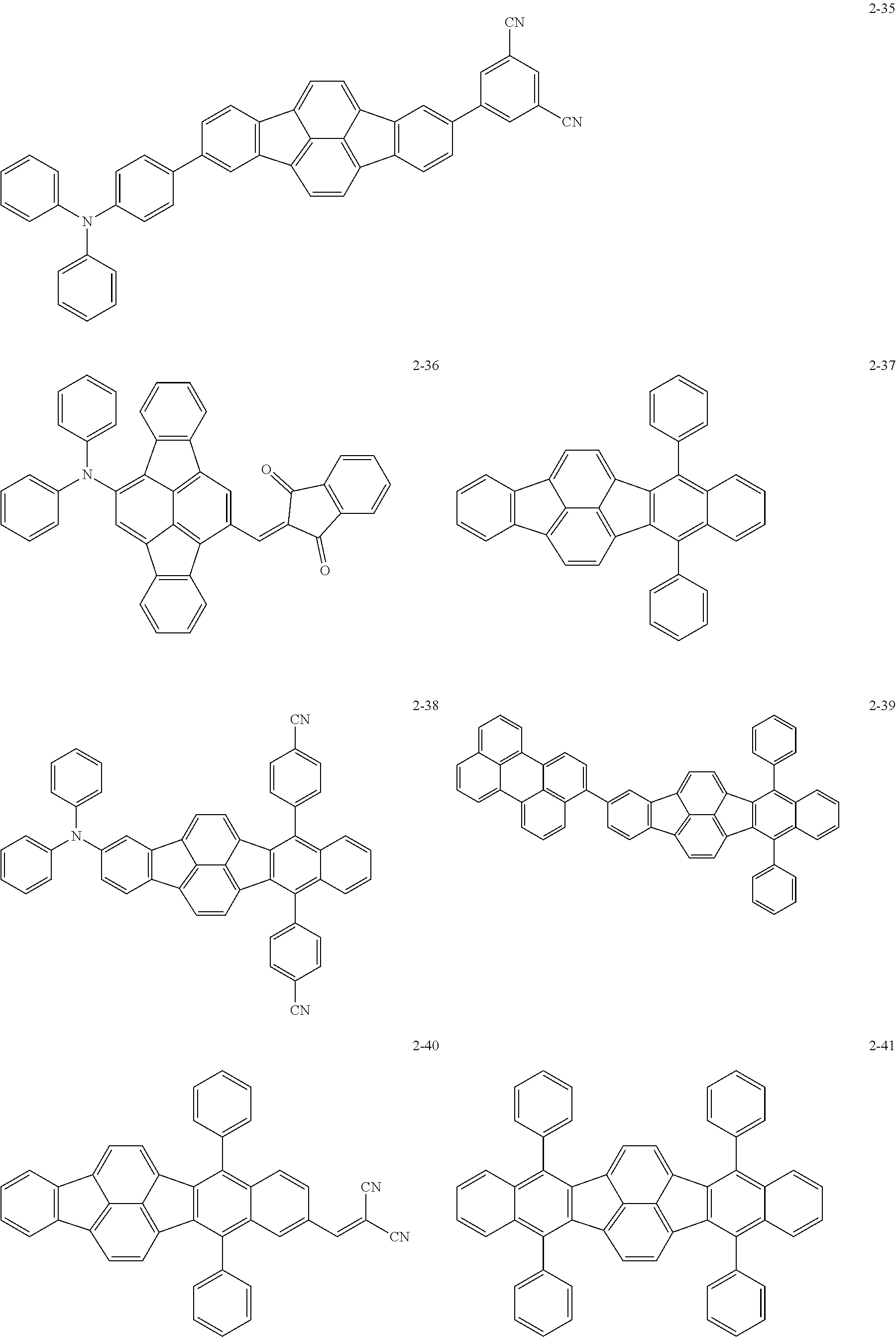

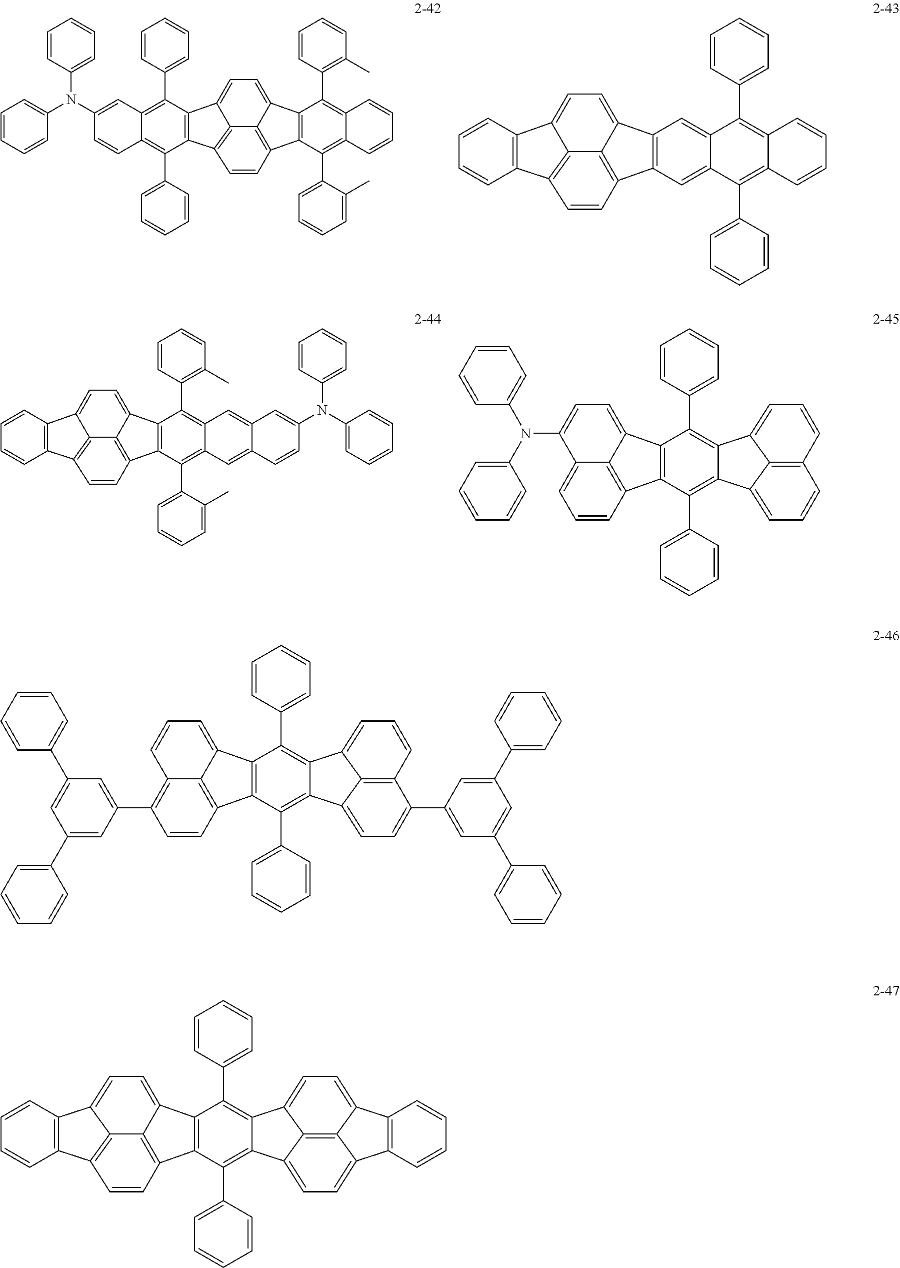

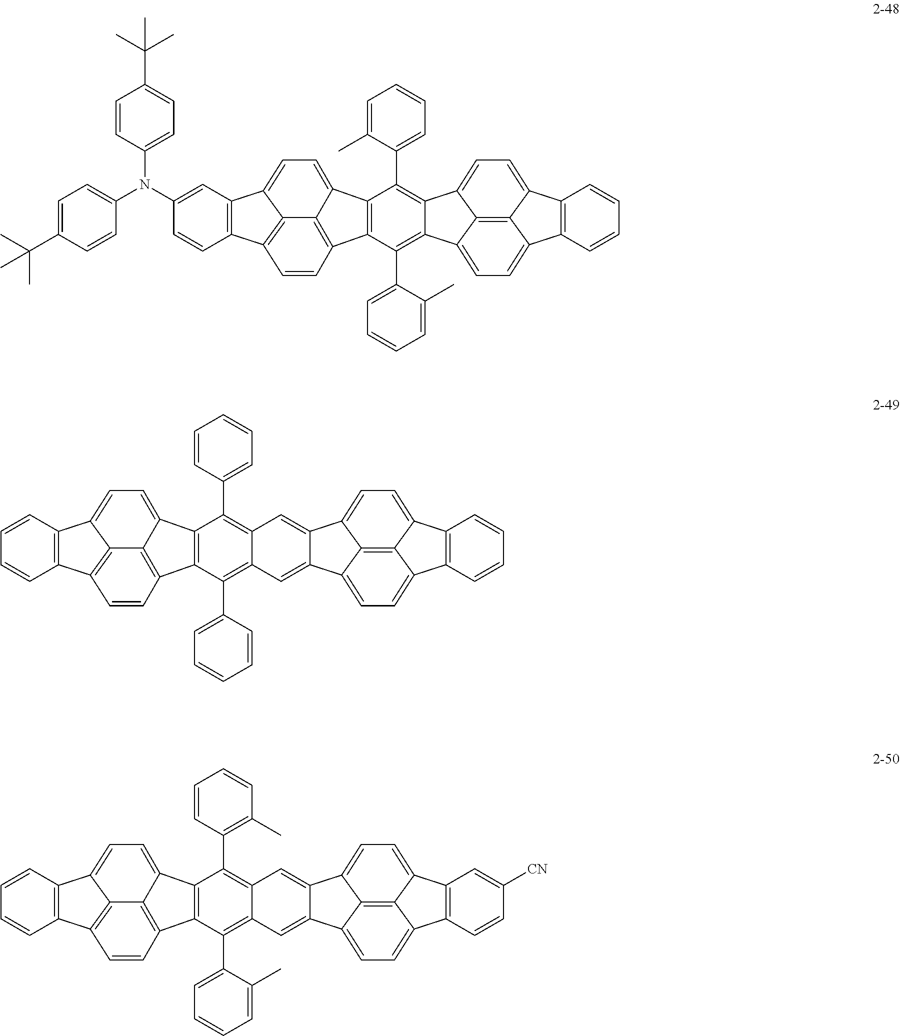

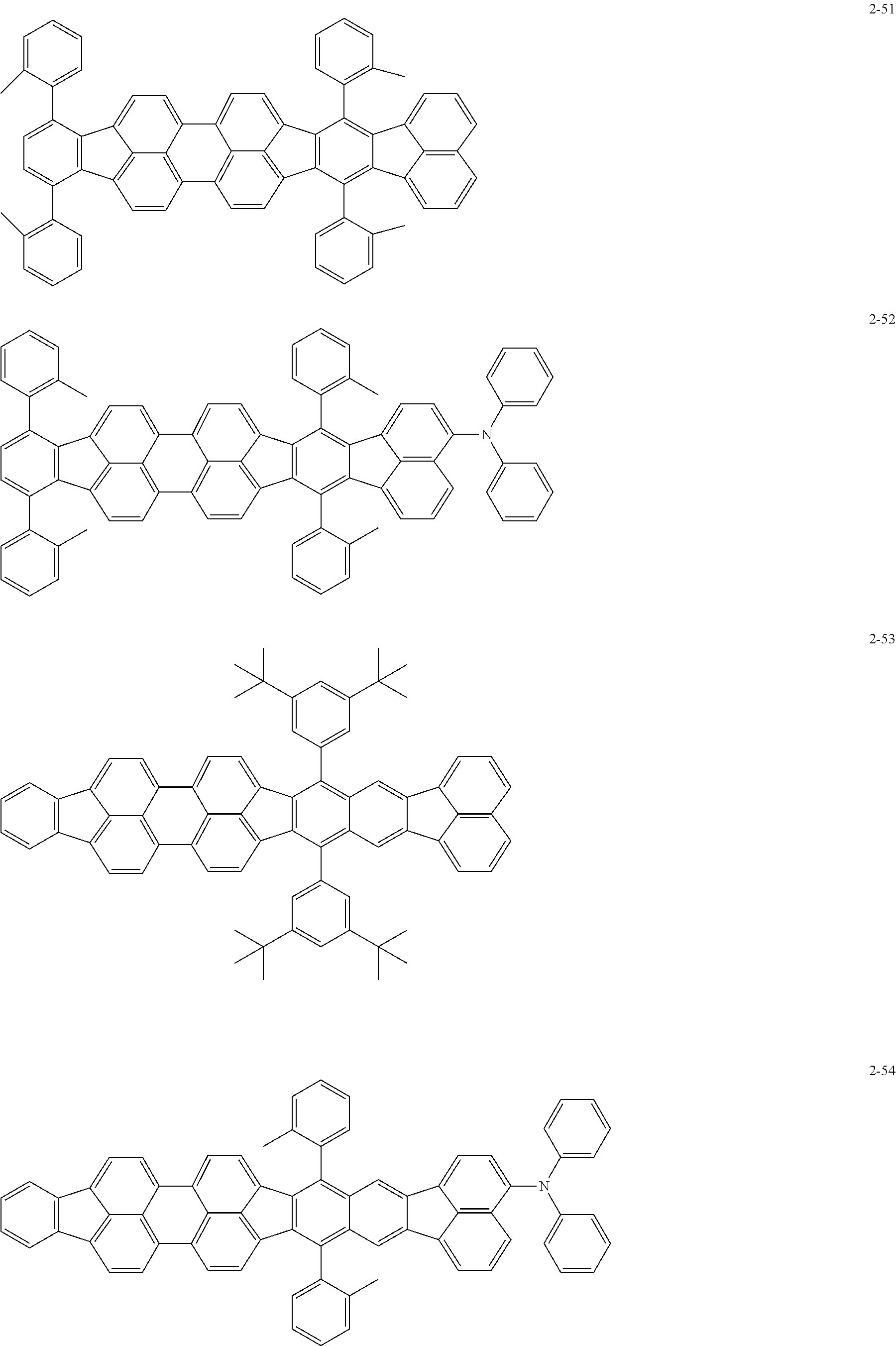

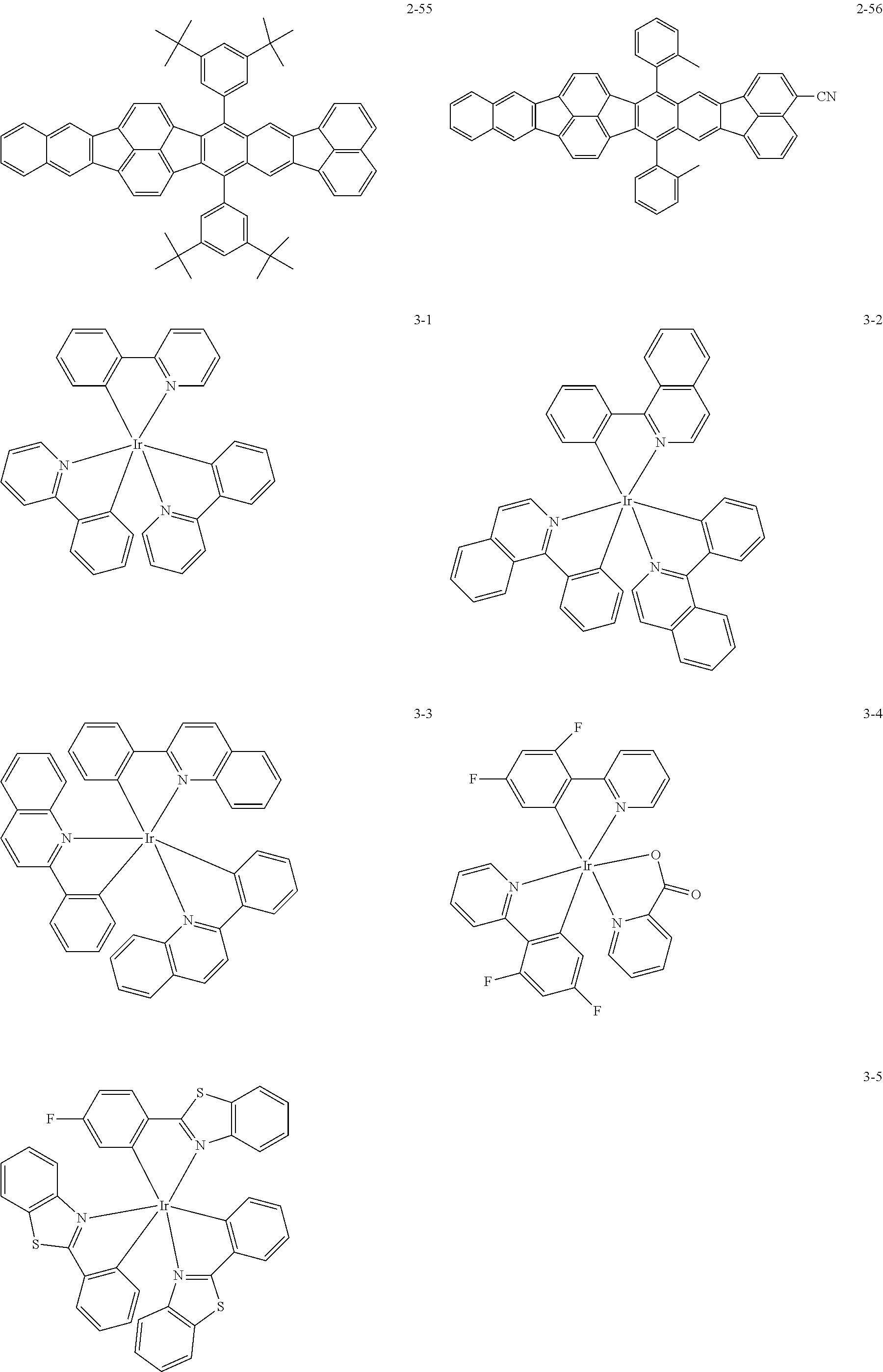

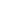

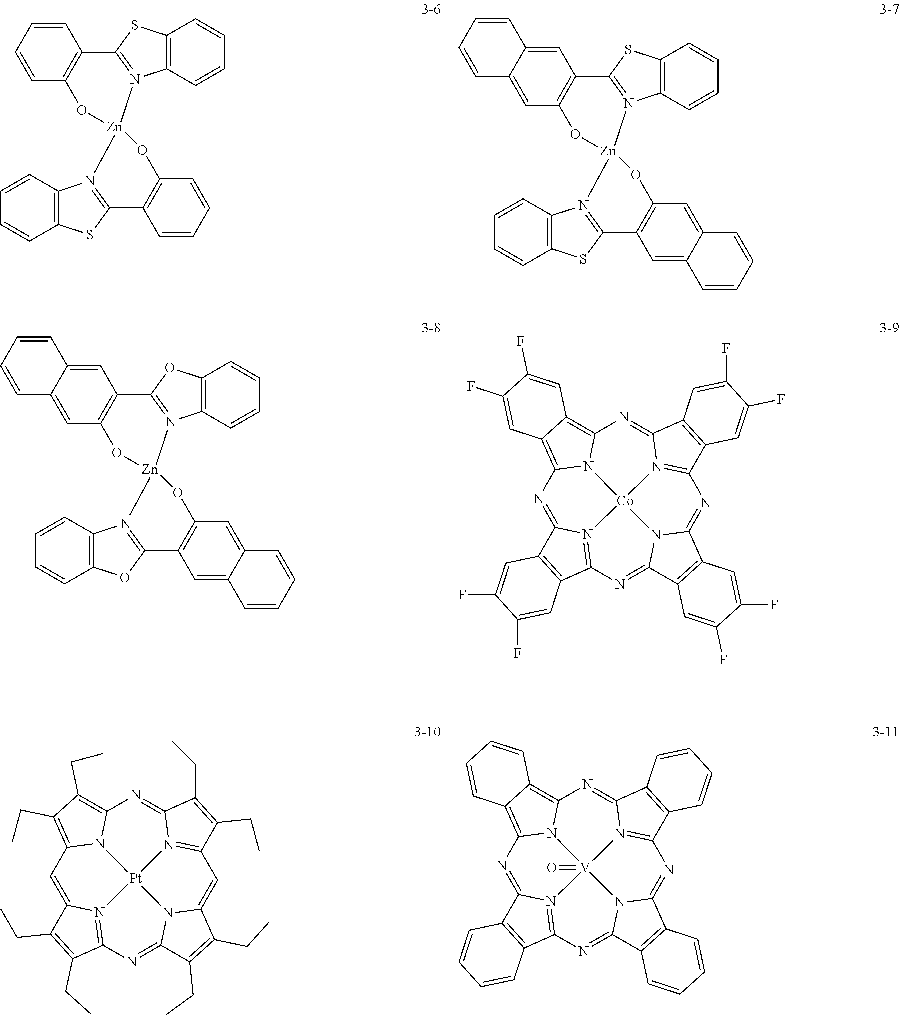

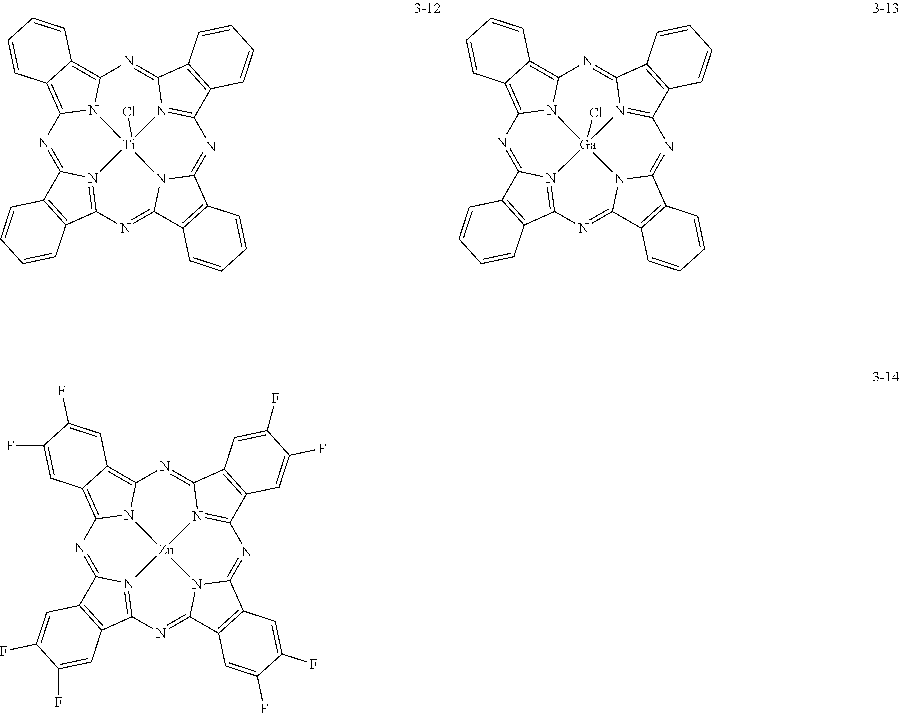

Example compounds 1-1 to 1-23 belong to a group of compounds having a sulfur-containing five-membered heterocyclic group at the center. The sulfur-containing five-membered heterocyclic group increases the absorption intensity in a longer wavelength region in the visible region and, as a result, can contribute to the panchromatic property of the photoelectric conversion layer. In addition, example compounds 1-1 to 1-23 exhibit long-lifetime emission and therefore preferred.

Example compounds 2-1 to 2-56 belong to a group of compounds having a fluoranthene skeleton at the center. Since the fluoranthene skeleton exhibits long-lifetime emission and has a low reduction potential, example compounds 2-1 to 2-56 are preferred as the first organic compound.

Example compounds 3-1 to 3-14 belong to a group of metal complex compounds. These metal complex compounds are phosphorescent compounds, and the exciton lifetime is longer than that of fluorescent organic compounds. Accordingly, example compounds 3-1 to 3-14 are preferred as the first organic compound.

Imaging Element According to Embodiment

The imaging element according to the embodiment includes a plurality of pixels, and each pixel includes the photoelectric conversion element according to the present invention and a readout transistor connected to the photoelectric conversion element.

The pixels may be arranged in a matrix including a plurality of rows and a plurality of columns. The pixels may be each connected to a signal processing circuit. The signal processing circuit receives a signal from each pixel to obtain an image.

The readout transistor is a transistor for transferring a signal based on the charge generated in the photoelectric conversion element.

The signal processing circuit may be a CMOS sensor or a CCD sensor.

The imaging element may include a light filter, for example, a color filter. When the photoelectric conversion element corresponds to light with a specific wavelength, the imaging element preferably includes a color filter corresponding to the photoelectric conversion element. One color filter may be provided for one light receiving pixel, or one color filter may be provided for multiple light receiving pixels.

Examples of the light filter include, in addition to the color filter, a low pass filter that transmits wavelengths equal to or longer than infrared light, and a UV cut filter that transmits wavelengths equal to or shorter than ultraviolet light.

The imaging element may include an optical member, such as a microlens. The microlens is a lens that collects light from the outside to the photoelectric conversion layer. One microlens may be provided for one light receiving pixel, or one microlens may be provided for multiple corresponding light receiving pixels. When a plurality of light receiving pixels is provided, it is preferable that each of the light receiving pixels is provided with one microlens.

The imaging element according to the present invention can be used in an imaging apparatus. The imaging apparatus includes an imaging optical system including a plurality of lenses and an imaging element receiving light passed through the imaging optical system. The imaging apparatus may include an imaging element and a housing accommodating the imaging element, and the housing may include a junction that can be connected to the imaging optical system. More specifically, the imaging apparatus is a digital camera or a digital still camera.

In addition, the imaging apparatus may further include a receiving unit that receives a signal from the outside. The signal received by the receiving unit is a signal that controls at least one of the imaging range of the imaging apparatus, start of imaging, and end of imaging. The imaging apparatus may further include a transmitting unit transmitting the acquired image to the outside. Examples of the acquired image include a captured image and an image transmitted from another device.

The imaging apparatus including a receiving unit and a transmitting unit can be used as a network camera.

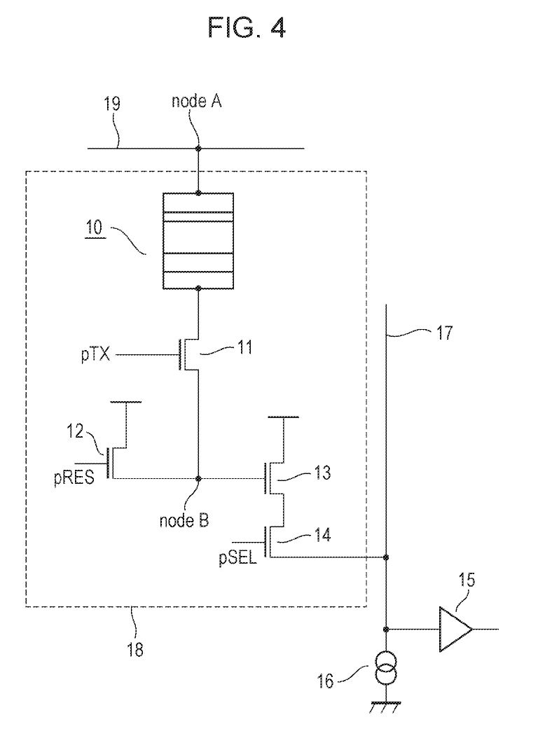

FIG. 4 is a circuit diagram illustrating an example of a pixel including a photoelectric conversion device according to the present invention. The photoelectric conversion device 10 is connected to a common wiring 19 at the node A. The common wiring may be grounded.

The pixel 18 may include a photoelectric conversion element 10 and a readout circuit for reading out the signal generated in the photoelectric conversion unit. The readout circuit may include, for example, a transfer transistor 11 electrically connected to the photoelectric conversion element, an amplification transistor 13 including a gate electrode electrically connected to the photoelectric conversion element 10, a selection transistor 14 selecting a pixel from which information is read out, and a reset transistor 12 supplying a reset voltage to the photoelectric conversion element.

The transfer by the transfer transistor 11 may be controlled by the pTX. The supply of a voltage by the reset transistor may be controlled by the pRES. The selection transistor is switched to the selection or non-selection state by the pSEL.

The transfer transistor 11, the reset transistor 12, and the amplification transistor 13 are connected to each other at the node B. In some configurations, the transfer transistor need not be provided.

The reset transistor supplies a voltage for resetting the potential of the node B. The pRES is applied to the gate of the reset transistor to control the supply of the voltage. In some configurations, the reset transistor need not be provided.

The amplification transistor supplies a current according to the potential of the node B. The amplification transistor is connected to the selection transistor 14 that selects the pixel outputting a signal. The selection transistor is connected to a current source 16 and a column output unit 15. The column output unit 15 may be connected to a signal processing unit.

The selection transistor 14 is connected to a vertical signal output line 17. The vertical signal output line 17 is connected to the current source 16 and the column output unit 15.

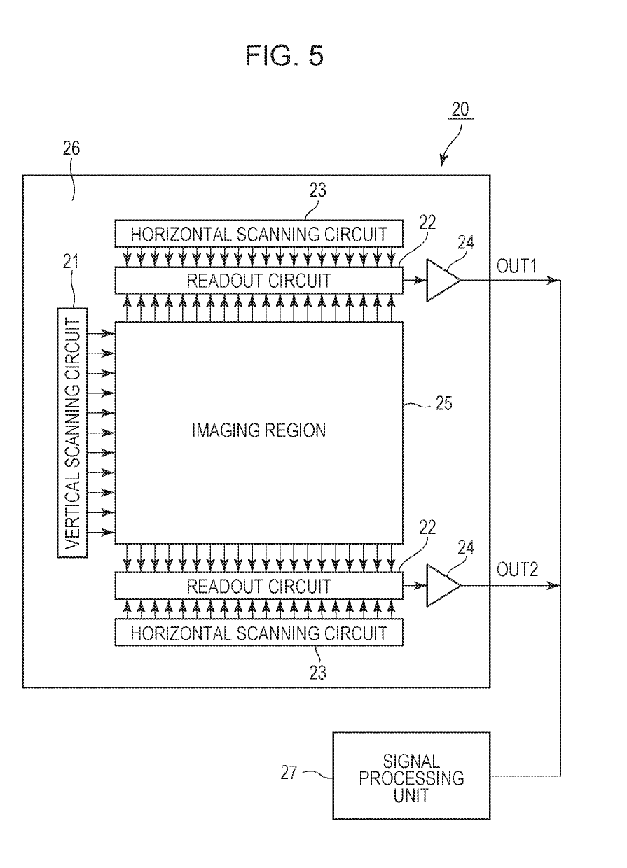

FIG. 5 is a diagram illustrating an imaging element according to the present invention and a peripheral circuit thereof. The imaging element 20 includes an imaging region 25 where a plurality of pixels are two-dimensionally arranged and a peripheral region 26. The region other than the imaging region is the peripheral region. The peripheral region includes a vertical scanning circuit 21, readout circuits 22, horizontal scanning circuits 23, and output amplifiers 24. The output amplifiers are connected to a signal processing unit 27. The signal processing unit processes signals by the information read out by the readout circuits and is, for example, a CCD circuit or a CMOS circuit.

The readout circuit 22 includes, for example, a column amplifier, a CDS circuit, and an adding circuit and performs, for example, amplification and addition of the signals read out from the pixels of the row selected by the vertical scanning circuit 21 through a vertical signal line. The column amplifier, the CDS circuit, the adding circuit, and so on are arranged, for example, for each pixel column or a plurality of pixel columns. The horizontal scanning circuit 23 generates signals for sequentially reading out the signals of the readout circuit 22. The output amplifier 24 amplifies and outputs the signals of the column selected by the horizontal scanning circuit 23.

The configurations described above are merely examples of the photoelectric conversion device, and the embodiment is not limited to them. The readout circuit 22, the horizontal scanning circuit 23, and the output amplifier 24 are arranged both above and below the imaging region 25 to constitute two output paths. However, the number of the output paths may be three. The signal output from each output amplifier is synthesized into an image signal at the signal processing unit.

### EXAMPLES

Examples of the present invention will now be described, but the present invention is not limited to the scope described in the examples.

Measurement of Emission Lifetime of First Organic Compound

The emission lifetime of the first organic compound in the present invention was measured with an apparatus having the following configuration.

Apparatus

Excitation light source: Picosecond light pulser (emission wavelength: 442 nm) manufactured by Hamamatsu Photonics K.K.

Spectroscope: Imaging spectrograph C5094 manufactured by Hamamatsu Photonics K.K.

Detector: Streak scope C4334 manufactured by Hamamatsu Photonics K.K.

Sample Preparation

Each compound was dissolved in chloroform, the concentration was adjusted such that the absorbance at a wavelength of 442 nm was about 0.05 to 0.2, and about 3 mL of the resulting solution was placed in a cell with an optical path length of 1 cm.

Measurement and Analysis of Emission Lifetime

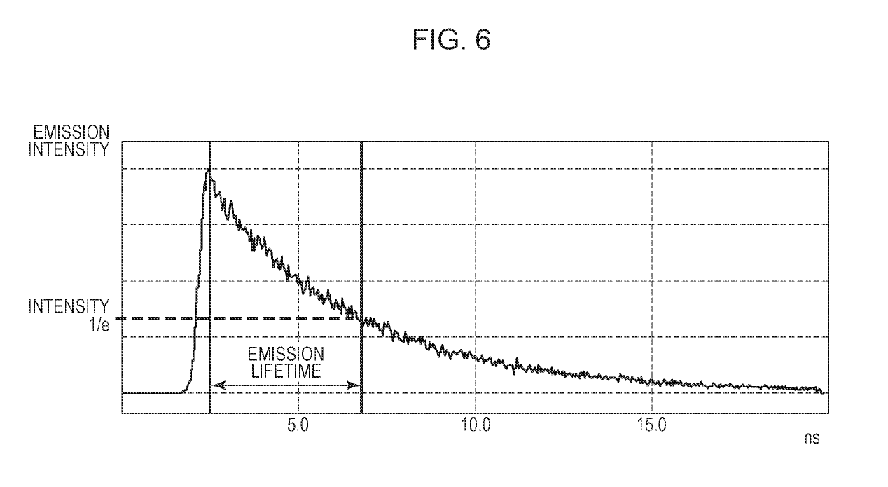

The sample solution was irradiated with excitation light with a wavelength of 442 nm to measure the time-resolved emission spectrum. FIG. 6 is a graph showing an example of a decay curve of the emission intensity. The decay curve was analyzed by one-component decay to obtain the emission lifetime. The emission lifetime was defined as the time until the initial intensity reached 1/e. Table 1 shows the emission lifetimes of example compounds of the first organic compound.

TABLE 1

| First organic | Emission lifetime in |
|---|---|
| compound | chloroform solution (ns) |
| 1-1 | 3.2 |
| 1-3 | 1.8 |
| 1-4 | 3.0 |
| 1-5 | 2.4 |
| 1-6 | 1.2 |
| 1-7 | 1.8 |
| 1-8 | 1.5 |
| 1-13 | 2.0 |
| 1-20 | 2.0 |
| 1-21 | 1.5 |
| 1-22 | 2.8 |
| 1-23 | 2.4 |
| 2-1 | 1.2 |
| 2-2 | 1.4 |
| 2-5 | 2.8 |
| 2-17 | 6.2 |
| 2-25 | 9.2 |
| 2-41 | 4.7 |
| 2-43 | 7.0 |
| 3-1 | 2000 |
| 3-2 | 1900 |

Measurement of Reduction Potential of First Organic Compound

Electrochemical characteristics, such as redox potential, can be evaluated by cyclic voltammetry (CV). The CV measurement sample was prepared by dissolving about 1 mg of a first organic compound in 10 mL of an ortho-dichlorobenzene solution containing 0.1 M tetrabutyl ammonium perchlorate and deaerating the solution with nitrogen. The measurement was performed by a three-electrode method using a nonaqueous solvent-type Ag/Ag⁺ reference electrode, a platinum counter electrode of 0.5 mm diameter and 5 cm length, and a glass carbon working electrode of 3 mm inner diameter (all manufactured by BAS Inc.). As the apparatus, an electrochemical analyzer model 660C manufactured by ALS was used. The sweep speed of the measurement was set to 0.1 V/s. Table 2 shows the reduction potentials of example compounds of the first organic compound.

TABLE 2

| First organic | Reduction potential in |
|---|---|
| compound | ortho-dichlorobenzene [V] |
| 1-1 | −1.23 |
| 1-3 | −1.15 |
| 1-4 | −1.17 |
| 1-5 | −1.18 |
| 1-6 | −1.24 |
| 1-7 | −1.24 |
| 1-8 | −1.15 |
| 1-13 | −1.13 |
| 1-20 | −1.21 |
| 1-21 | −1.20 |
| 1-22 | −1.14 |
| 1-23 | −1.32 |
| 2-1 | −1.22 |
| 2-2 | −1.08 |
| 2-5 | −1.69 |
| 2-17 | −1.84 |
| 2-25 | −1.90 |
| 2-41 | −1.49 |
| 2-43 | −1.50 |
| 3-1 | −2.38 |
| 3-2 | −2.02 |

### Example 1

In this example, a photoelectric conversion element including a first organic compound having an emission lifetime of 1.1 ns or more in chloroform solution and a second organic compound was produced. The element characteristics of the produced element were evaluated.

In the example, a photoelectric conversion element was formed on a Si substrate. The photoelectric conversion element includes a cathode electrode, an electron blocking layer, a photoelectric conversion layer, a hole blocking layer, and an anode electrode formed in this order.

In the example, the photoelectric conversion element was produced by the following steps.

Prepared first was a Si substrate on which a wiring layer and an insulating layer are laminated and openings were provided in the insulating layer at positions corresponding to pixels to form contact holes so as to be conductive from the wiring layer. The contact holes were connected to the pad portion at the substrate end by wiring. An IZO electrode was formed so as to overlap the contact hole portion by sputtering, followed by patterning to form an IZO electrode (cathode electrode) of 3 mm². On this occasion, the IZO electrode had a thickness of 100 nm.

On the IZO electrode, an organic compound layer was formed by a vacuum vapor deposition method. The layer composition and thickness were as shown in Table 3. Next, IZO was formed, as an anode electrode, by sputtering. The anode electrode had a thickness of 30 nm.

The layer composition of the photoelectric conversion element is shown in Table 3.

TABLE 3

| | Constituent material | Thickness | |
|---|---|---|---|
| Anode electrode | IZO | 30 | nm |
| Hole blocking layer | d-2(C60) | 20 | nm |
| Photoelectric | Example compound | 380 | nm |
| conversion layer | 1-6:d-2(C60) = 25:75 | | |
| Electron blocking layer | d-1 | 80 | nm |
| Cathode electrode | IZO | 100 | nm |

In Table 3, the cathode as the lower electrode is shown on the lower side of the table.

The electron blocking layer was formed of the following compound (d-1).

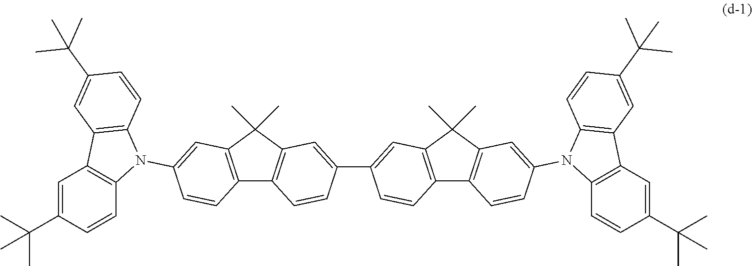 [SMILES: CC(C)(C)c1ccc2c(c1)c1cc(C(C)(C)C)ccc1n2-c1ccc2c(c1)C(C)(C)c1cc(-c3ccc4c(c3)C(C)(C)c3cc(-n5c6ccc(C(C)(C)C)cc6c6cc(C(C)(C)C)ccc65)ccc3-4)ccc1-2]

As the first organic compound of the photoelectric conversion layer, any one of example compounds 1-1 to 3-14 was used, and as the material for the hole blocking layer, any one of fullerene C60 (d-2), C70 (d-3), and the following organic compound (d-4) was used.

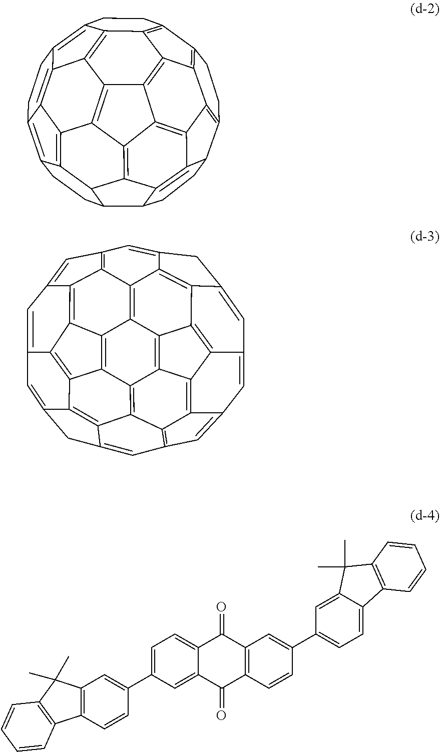

The reduction potentials of compounds d-2, d-3, and d-4 were as shown in Table 4.

TABLE 4

| Second organic | |
|---|---|
| compound | Ered (V) |
| d-2 | −0.83 |
| d-3 | −0.79 |
| d-4 | −1.04 |

After formation of the upper electrode, hollow sealing was performed using a glass cap and an ultraviolet curing resin. The thus-obtained element was annealed on a hot plate at 170° C. for about 1 hour with the sealing face facing upward.

The characteristics of the resulting photoelectric conversion elements were measured and evaluated. A voltage of 5 V was applied to each element to verify the current, and it was observed that the current value in a bright place was 100 times or more the current value in a dark place in every element, which demonstrated that the photoelectric conversion elements were functioning.

The external quantum efficiency of the resulting element was calculated by measuring the density of the photocurrent that flew when the produced element was irradiated with monochromatic light having a wavelength of 550 nm (green light) and an intensity of 50 μW/cm² in a state in which a voltage of 5 V was applied between the cathode electrode and the anode electrode.

The photocurrent density was determined by subtracting the dark current density at light shielding time from the current density at light irradiation time. The monochromatic light used for the measurement was obtained by monochromating white light emitted from a xenon lamp (device name: XB-50101AA-A, manufactured by Ushio Inc.) with a monochrometer (device name: MC-10N, manufactured by Ritu Oyo Kougaku Co., Ltd.). The voltage application to an element and the current measurement were performed using a source meter (device name: R6243, manufactured by Advantest Corporation). The produced photoelectric conversion element was irradiated with light perpendicular to the electrode from the upper electrode side.

The external quantum efficiency determined as described above is affected by the light absorptivity of the organic compound. The light absorptivity of the organic compound varies depending on the type of the compound. Accordingly, in order to reduce the influence, the efficiency of the photoelectric conversion element was evaluated by the photoelectric conversion efficiency represented by the following Expression (C).

Photoelectric conversion efficiency=(external quantum efficiency)/absorptivity (C)

Here, the absorptivity was measured with SolidSpec-3700 UV-VIS-NIR-Spectrophotometer manufactured by Shimadzu Corporation. The measurement was performed by producing a sample in which a film having the same configuration as that of the photoelectric conversion layer was formed on a quartz substrate and determining the absorptivity of this film.

The results of evaluation of the photoelectric conversion efficiency are shown in Table 6 together with the results of other examples.

### Examples 2 to 22, Comparative Examples 1 to 9

Photoelectric conversion elements were produced as in Example 1 except that the combination of organic compounds included in the photoelectric conversion layer was that shown in Table 6, and the photoelectric conversion efficiency was evaluated. In Examples 17 to 22, a phosphorescence-emitting material was used as the first organic compound layer.

The organic compounds e-1 to e-3 used in Comparative Examples 1 to 9 are organic compounds represented by the following Formulae.

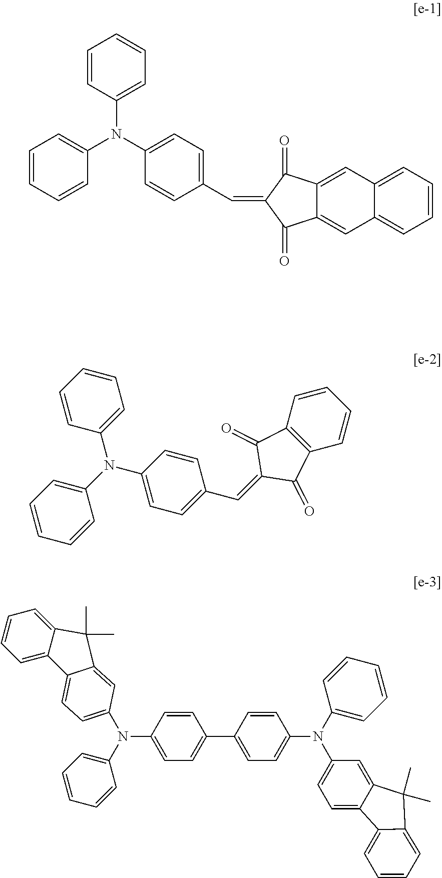 [SMILES: O=C1C(=Cc2ccc(N(c3ccccc3)c3ccccc3)cc2)C(=O)c2cc3ccccc3cc21 ; O=C1C(=Cc2ccc(N(c3ccccc3)c3ccccc3)cc2)C(=O)c2ccccc21 ; CC1(C)c2ccccc2-c2ccc(N(c3ccccc3)c3ccc(-c4ccc(N(c5ccccc5)c5ccc6c(c5)C(C)(C)c5ccccc5-6)cc4)cc3)cc21]

The emission lifetimes in chloroform solution and reduction potentials in ortho-dichlorobenzene of organic compounds e-1 to e-3 are shown in Table 5.

TABLE 5

| | Emission lifetime in | Reduction potential in |
|---|---|---|
| Compound | chloroform solution (ns) | ortho-dichlorobenzene (V) |
| e-1 | 1.0 | −1.24 |
| e-2 | 0.6 | −1.28 |
| e-3 | 1.0 | −2.61 |

The results of Examples 1 to 22 and Comparative Examples 1 to 9 are shown in Table 6. The evaluation criteria of the photoelectric conversion efficiency are as follows:

A: 75% or more,

B: 65% or more and less than 75%, and

C: less than 65%.

Here, judgement as B or higher was decided as good, and judgement as C was decided as bad.

TABLE 6

| | | | Emission | | |
|---|---|---|---|---|---|
| | | | lifetime | | |
| | | | of first | | |
| | First | Second | organic | | |
| | organic | organic | compound | ΔEred | |
| Example | compound | compound | (ns) | (V) | Evaluation |
| 1 | 1-6 | d-2 | 1.2 | 0.41 | B |
| 2 | 1-6 | d-3 | 1.2 | 0.45 | B |
| 3 | 1-7 | d-2 | 1.8 | 0.41 | A |
| 4 | 1-7 | d-3 | 1.8 | 0.45 | B |
| 5 | 1-20 | d-2 | 2.0 | 0.38 | A |
| 6 | 1-20 | d-3 | 2.0 | 0.42 | B |
| 7 | 1-21 | d-2 | 1.5 | 0.37 | A |
| 8 | 1-21 | d-3 | 1.5 | 0.41 | B |
| 9 | 1-23 | d-2 | 2.4 | 0.49 | A |
| 10 | 1-23 | d-3 | 2.4 | 0.53 | A |
| 11 | 2-1 | d-2 | 1.2 | 0.39 | A |
| 12 | 2-1 | d-3 | 1.2 | 0.43 | B |
| 13 | 2-25 | d-2 | 9.2 | 1.07 | B |
| 14 | 2-25 | d-3 | 9.2 | 1.11 | B |
| 15 | 2-41 | d-2 | 4.7 | 0.66 | B |
| 16 | 2-41 | d-3 | 4.7 | 0.70 | B |
| 17 | 3-1 | d-2 | 2000 | 1.55 | B |
| 18 | 3-1 | d-3 | 2000 | 1.59 | B |
| 19 | 3-1 | d-4 | 2000 | 1.34 | B |
| 20 | 3-2 | d-2 | 1900 | 1.19 | B |
| 21 | 3-2 | d-3 | 1900 | 1.23 | B |
| 22 | 3-2 | d-4 | 1900 | 0.98 | B |
| Comparative | e-1 | d-2 | 1.0 | 0.41 | C |
| Example 1 | | | | | |
| Comparative | e-1 | d-3 | 1.0 | 0.45 | C |
| Example 2 | | | | | |
| Comparative | e-1 | d-4 | 1.0 | 0.20 | C |
| Example 3 | | | | | |
| Comparative | e-2 | d-2 | 0.6 | 0.45 | C |
| Example 4 | | | | | |
| Comparative | e-2 | d-3 | 0.6 | 0.49 | C |
| Example 5 | | | | | |
| Comparative | e-2 | d-4 | 0.6 | 0.24 | C |
| Example 6 | | | | | |
| Comparative | e-3 | d-2 | 1.0 | 1.78 | C |
| Example 7 | | | | | |
| Comparative | e-3 | d-3 | 1.0 | 1.82 | C |
| Example 8 | | | | | |
| Comparative | e-3 | d-4 | 1.0 | 1.57 | C |
| Example 9 | | | | | |

When the emission lifetime of the first organic compound was 1.1 ns or more, the photoelectric conversion efficiency was evaluated as B or higher in every combination of compounds in the photoelectric conversion elements. Furthermore, when the emission lifetime of the first organic compound was 1.1 ns or more and ΔEred≥0.32 was satisfied, the photoelectric conversion efficiency was further higher.

In particular, in the range of satisfying 0.32≤ΔEred≤0.65, high photoelectric conversion efficiencies were obtained. An organic compound having a ΔEred of higher than 0.65 has a low oxidation potential. Consequently, the dark current of the photoelectric conversion element tends to increase.

In contrast, in Comparative Examples 1 to 9 using an organic compound having an emission lifetime of less than 1.1 ns as the first organic compound, the photoelectric conversion efficiency was low, which demonstrates that the emission lifetime of the first organic compound of 1.1 ns or more is effective for high efficiency.

Based on the results above, a photoelectric conversion element with high efficiency can be provided by using a first organic compound having an absorption peak wavelength in the visible region and having an emission lifetime in chloroform solution of 1.1 ns or more.

The present invention is not limited to the above-described embodiments, and various modifications and variations are possible without departing from the spirit and scope of the present invention. Accordingly, in order to publicize the scope of the present invention, the following claims are attached.

According to the present invention, it is provided a photoelectric conversion element having high photoelectric conversion efficiency in the visible light region by using a photoelectric conversion layer including a first organic compound having an exciton lifetime of at least a certain length.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

1. A photoelectric conversion element comprising a lower electrode, a photoelectric conversion layer, and an upper electrode in this order, wherein
the photoelectric conversion layer includes a first organic compound and a second organic compound having a lower reduction potential than that of the first organic compound;
the first organic compound has an emission lifetime of 1.1 ns or more in chloroform solution; and
the first organic compound is an organic compound represented by any one of the following Formulae [1] to [5]: 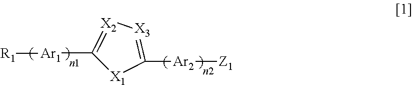
in Formula [1], R₁ represents a hydrogen atom, a halogen atom, a substituted or unsubstituted alkyl group, a substituted or unsubstituted alkoxy group, a substituted or unsubstituted aryl group, a substituted or unsubstituted heterocyclic group, a substituted or unsubstituted vinyl group, a substituted or unsubstituted amino group, or a cyano group;
n1 and n2 each represent an integer of 0 to 4;
X₁ to X₃ each represent a nitrogen atom, a sulfur atom, an oxygen atom, or a carbon atom, and the carbon atom optionally has a substituent;
Ar₁ and Ar₂ are each independently selected from the group consisting of a substituted or unsubstituted aryl group and a substituted or unsubstituted heterocyclic group, when there is more than one Ar₁, Ar₁'s are the same or different and there is more than one Ar₂, Ar₂'s are the same or different, and when X₂ or X₃ is a carbon atom, Ar₁ and Ar₂ optionally form a ring by bonding to the carbon atom; and
Z₁ represents a halogen atom, a cyano group, a substituted or unsubstituted heteroaryl group, or substituent represented by any one of the following Formulae [1-1] to [1-9], in Formulae [1-1] to [1-9], R₅₂₁ to R₅₈₈ are each independently selected from the group consisting of a hydrogen atom, a halogen atom, a substituted or unsubstituted alkyl group, a substituted or unsubstituted alkoxy group, a substituted or unsubstituted aryl group, a substituted or unsubstituted heterocyclic group, a substituted or unsubstituted vinyl group, a substituted or unsubstituted amino group, and a cyano group, 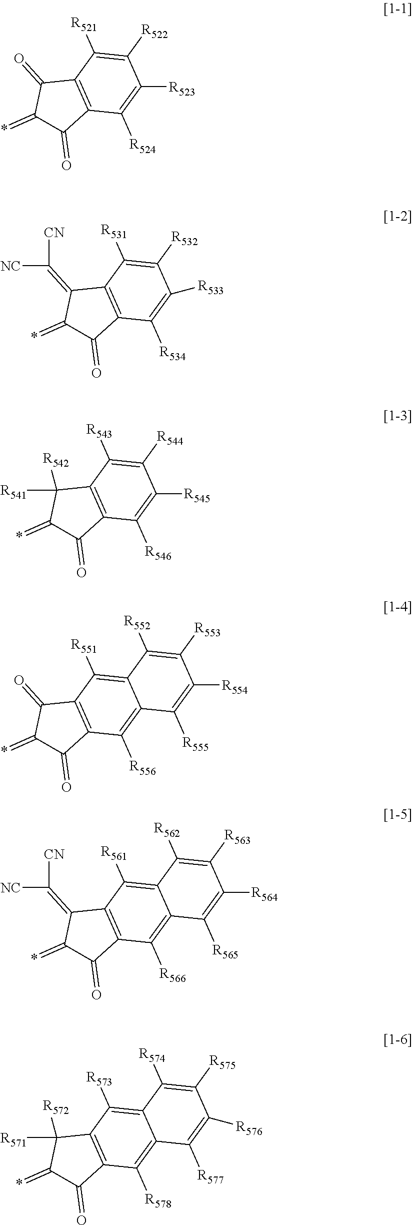 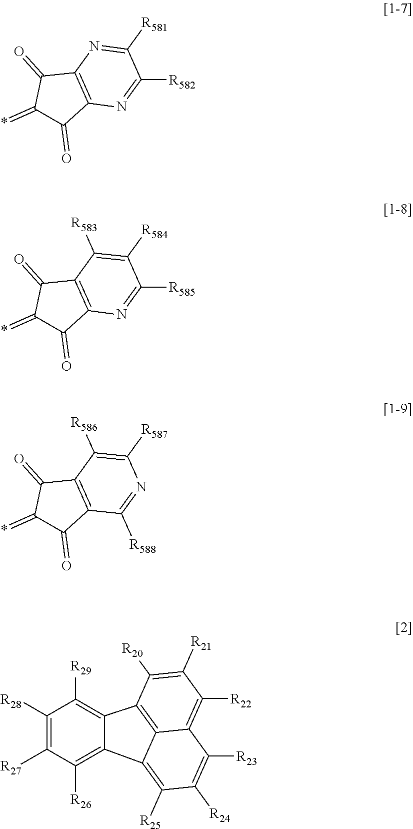
in Formula [2], R₂₀ to R₂₉ are each independently selected from the group consisting of a hydrogen atom, a halogen atom, a substituted or unsubstituted alkyl group, a substituted or unsubstituted alkoxy group, a substituted or unsubstituted aryl group, a substituted or unsubstituted heterocyclic group, a substituted or unsubstituted vinyl group, a substituted or unsubstituted amino group, and a cyano group, where adjacent two of R₂₀ to R₂₉ optionally form a ring by bonding to each other and to form a compound represented by any one of the following Formulae [11], [13] to [21], and [23] to [27]: 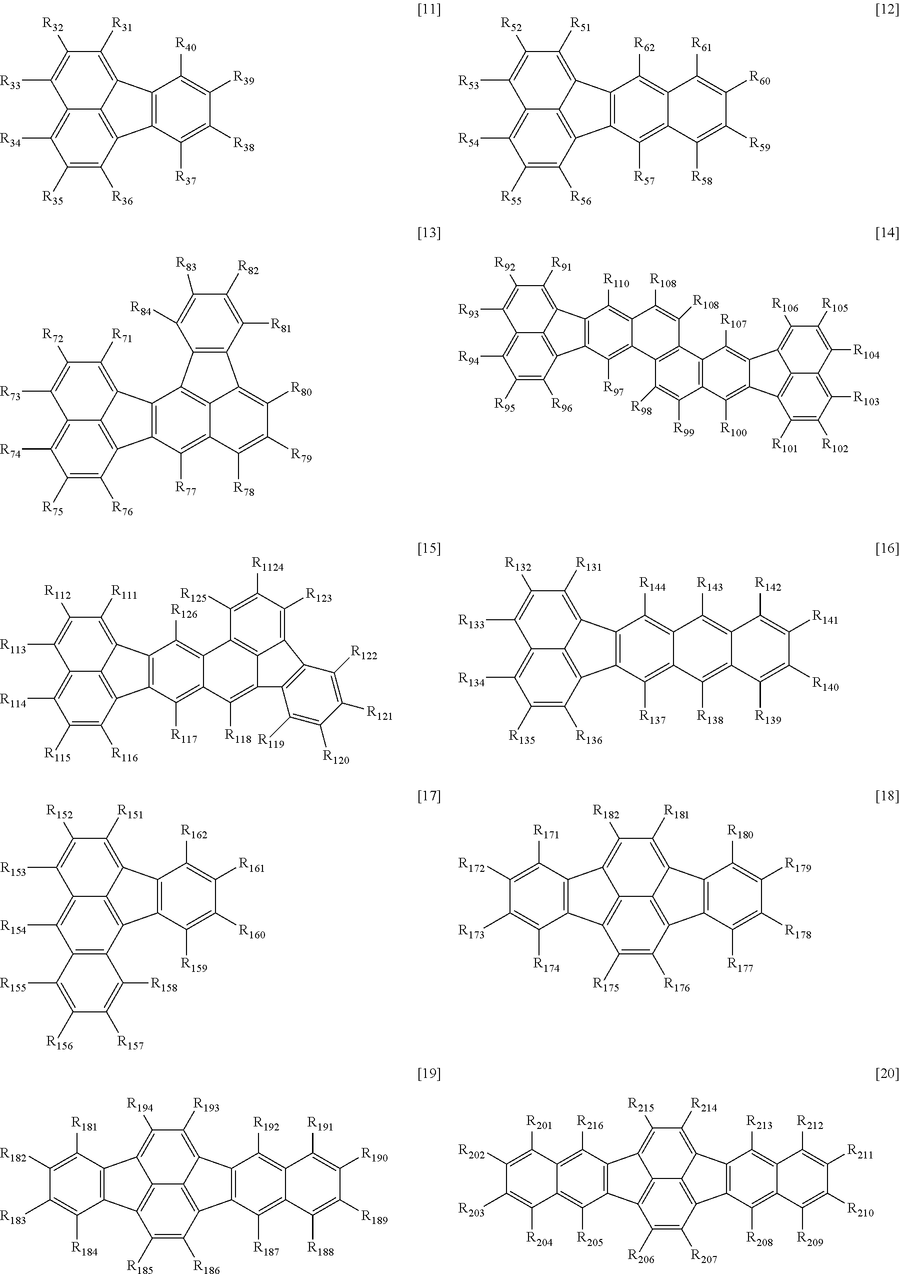 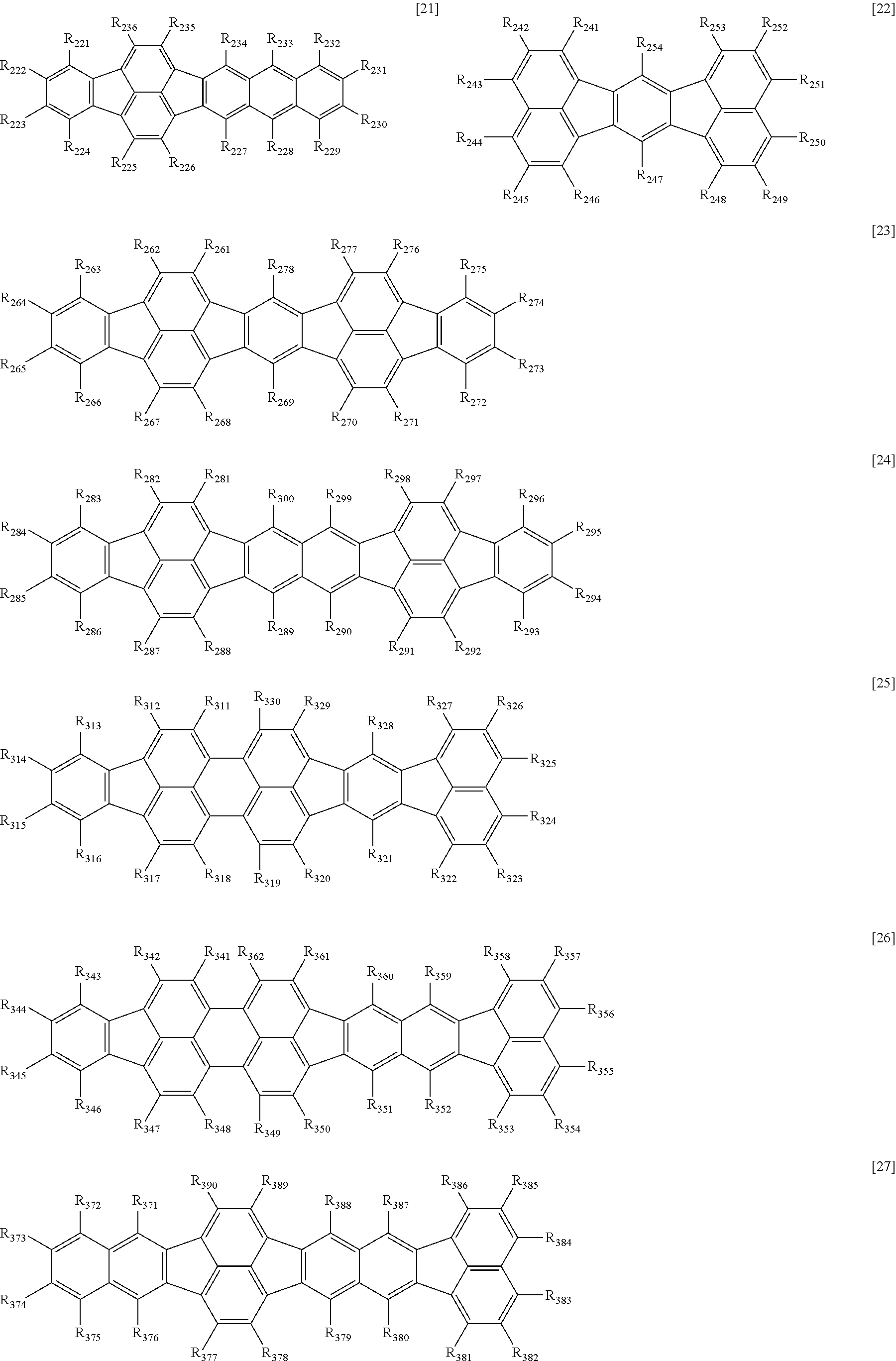
in Formulae [11], [13] to [21], and [23] to [27], R₃₁ to R₃₉₀ are each independently selected from the group consisting of a hydrogen atom, a halogen atom, a substituted or unsubstituted alkyl group, a substituted or unsubstituted alkoxy group, a substituted or unsubstituted aryl group, a substituted or unsubstituted heterocyclic group, a substituted or unsubstituted vinyl group, a substituted or unsubstituted amino group, and a cyano group, 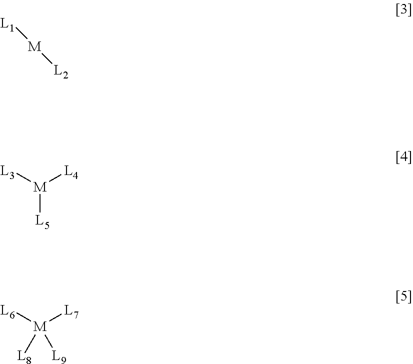
in Formulae [3] to [5], M represents a metal atom, where the metal atom optionally has an oxygen atom or a halogen atom as a substituent; and
L₁ to L₉ each represent a ligand coordinating to metal M, where the ligands are each a substituted or unsubstituted aryl group or a substituted or unsubstituted heterocyclic group, and adjacent two of L₁ to L₉ optionally form a ring by bonding to each other.

2. The photoelectric conversion element according to claim 1, wherein in Formula [1], Ar₁ represents an aryl group or a heterocyclic group; X₁ represents a sulfur atom or an oxygen atom; n1 is 1; and n2 is 0.

3. The photoelectric conversion element according to claim 1, wherein in Formulae [3] to [5], M represents iridium, platinum, vanadium, cobalt, gallium, or titanium.

4. The photoelectric conversion element according to claim 1, wherein in the photoelectric conversion layer, ΔEred represented by the following Expression (A) satisfies the following Expression (B):
ΔEred=(reduction potential of second organic compound)−(reduction potential of first organic compound) (A),
ΔEred≥0.32 [V] (B).

5. The photoelectric conversion element according to claim 4, wherein the photoelectric conversion layer satisfies the following Expression (C):
0.32≤ΔEred≤0.65 [V] (C).

6. The photoelectric conversion element according to claim 1, wherein the second organic compound is a fullerene derivative.

7. The photoelectric conversion element according to claim 6, wherein the fullerene derivative is fullerene C60.

8. The photoelectric conversion element according to claim 1, further comprising a sealing layer on the upper electrode.

9. An imaging element comprising:
a plurality of pixels; and
a signal processing circuit connected to the pixels, wherein
the pixels each include the photoelectric conversion element according to claim 1 and a readout circuit connected to the photoelectric conversion element.

10. An imaging apparatus comprising:
an optical unit including a plurality of lenses; and
an imaging element receiving light passed through the optical unit, wherein
the imaging element is the imaging element according to claim 9.

11. The imaging apparatus according to claim 10, further comprising a receiving unit receiving a signal from outside.

12. The imaging apparatus according to claim 11, wherein the signal is a signal controlling at least one of the imaging range of the imaging apparatus, start of imaging, and end of imaging.

13. The imaging apparatus according to claim 10, further comprising a transmitting unit transmitting an acquired image to outside.
